(19) United States
(12) Patent Application Publication
(10) Pub. No.: US 2021/0023259 A1
(43) Pub. Date: Jan. 28, 2021

(54) POLY (IONIC LIQUID) COMPOSITIONS AND THEIR USE AS TISSUE ADHESIVES

(71) Applicant: Rowan University, Glassboro, NJ (US)

(72) Inventor: Iman Noshadi, Haddon Heights, NJ (US)

(21) Appl. No.: 17/044,591

(22) PCT Filed: Apr. 2, 2019
(86) PCT No.: PCT/US2019/025431

§ 371 Date: Oct. 1, 2020

Related U.S. Application Data
(60) Provisional application No. 62/651,514, filed on Apr. 2, 2018.

(51) Int. Cl. A61L 24/04; A61L 24/00; A61B 17/00

(52) U.S. Cl. CPC A61L 24/043; A61L 24/0031; A61L 24/001; A61B 2017/00495; A61L 2400/06; A61L 2400/04; A61B 2017/005; A61B 17/00491

(57) ABSTRACT

The present invention relates to the discovery of methods of treating a wound in a subject in need thereof. In certain embodiments, the method comprises contacting the wound with a composition comprising gelatin methacrylate and choline acrylate, and then polymerizing the composition to form a polymerized composition having a plurality of choline acrylate functionalized gelatin methacrylate units.

### CROSS-REFERENCE TO RELATED APPLICATIONS

The present application claims priority under 35 U.S.C. § 119(e) to U.S. Provisional Application No. 62/651,514, filed Apr. 2, 2018, which is hereby incorporated by reference herein in its entirety.

### BACKGROUND OF THE INVENTION

Soft tissues and skeletal muscles are often involved in traumatic injury in cases of accidents, gunshot wounds, and blast injuries. Death rates are high for patients involved in severe trauma due to complications associated with such injuries during the early stages of trauma. For example, an estimated 80% of military deaths in such cases are caused by severe hemorrhagic loss of blood. Thus, severe trauma management should entail the cessation of bleeding and prevention of excessive loss of body fluids. Further, injured sites should be protected from pathogenic attack and infection, especially in septic conditions, by employing proper coverage.

Stable blood clot formation, also known as hemostasis, is the essential basis for preventing blood loss and hence death from excessive bleeding. The body's natural coagulation process is divided into primary hemostasis and coagulation cascade. These processes convert the blood into stable and insoluble fibrin to enable hemostasis. In cases of severe trauma, however, the organic rate of hemostasis, without assistance from external hemostatic devices and agents, is not rapid enough to prevent excessive loss of blood. Traditionally, compression using gauze and suturing the wounded tissues have been employed to achieve hemostasis. However, these traditional methods are rather ineffective in managing massive bleeding, and especially controlling hemorrhage involved in cardiovascular, liver, kidney, orthopedic, and spinal procedures. Additional continuous bleeding by oozing may also lead to hemorrhagic shock or death if not mitigated by proper hemostatic treatment.

The primary requirements of a good hemostatic material for in vivo application are the ability to rapidly hold blood and to adhere quickly and strongly to the tissues. Attachment and adhesion to the surrounding tissue is often difficult, particularly in the wet and dynamic environment often encountered in massive bleeding scenarios. The adhesive patch is also expected to degrade in vivo and have no cytotoxicity (and generate no cytotoxic degradation products). Thus, an ideal adhesive patch should accelerate healing without having any deleterious influence on the healing of the wound. The quality, cost of manufacture, stability in vivo, and safety of the adhesive patch are further considerations. Mechanical compliance and tunable adhesion can also be essential characteristics.

For such an application, to date, studies have been conducted on a variety of biologically-based materials as topical hemostatic agents. Such biologically-based materials include fibrinogen, fibrin, albumin, thrombin, collagen, gelatin, chitosan, cellulose, starch, and alginate as examples. Effective hemostatic materials with adhesive and antimicrobial properties have also been synthesized using isocyanate, poly(ethylene glycol) (PEG) and catechol containing monomers. Inorganic materials such as mineral zeolite, kaolin, smectite and bioactive glass have also been studied in hemostatic applications.

While a number of hemostatic materials are commercially available, each possesses one or more issues concerning biosafety, cost, efficiency, and rapidity of effecting hemostasis. The biologically derived hemostatic agents entail high cost, short shelf life, and potential risk of pathogenic contamination. Synthetic materials exhibit issues involving cytotoxicity and non-biodegradability. Inorganic materials have been shown to cause thermal injuries, in addition to inflammation, due to exothermic reactions in vivo, while also suffering from poor biodegradability in clinical applications.

Another important property for an effective hemostatic sealant is its antibacterial activity. Pathogenic bacteria such as Staphylococcus aureus, Pseudomonas aeruginosa, Streptococcus pyrogenes and some Proteus, Clostridium, and Coliform species can be detrimental to the healing process. Inadequate control measures to manage infected injuries can lead to cellulitis and ultimately bacteremia and septicemia, both of which can be fatal. About 50% of wounds slow to heal contain P. aeruginosa and S. aureus.

Current infection treatment strategies include oral and intravenous antibacterial administrations, which need much larger dose than would be required if administered locally. Further, systemic antibiotic administration can also have several side effects on healthy tissues or organs, and can lead to the dangerous rise in drug-resistant bacteria.

There remains a need in the art for effective hemostatic sealant materials. In certain embodiments, such materials are non-toxic, cheap and effective at sealing a wound while simultaneously preventing bacterial infection. The present invention meets this need.

### BRIEF SUMMARY OF THE INVENTION

The invention provides a method of treating a wound in a subject in need thereof. In certain embodiments, the method comprises contacting the wound with a composition comprising choline acrylate, a polymer selected from the group consisting of gelatin methacrylate (GelMa) and poly(ethylene glycol) diacrylate (PEGDA), and at least one photoinitiator. Subsequently, the method comprises exposing the composition to at least one wavelength of light capable of activating the at least one photoinitiator, thereby polymerizing the composition.

In certain embodiments, the composition comprises about 1:4 to about 4:1 choline acrylate to polymer. In certain other embodiments, the composition comprises about 1:1 choline acrylate to polymer.

In certain embodiments, the at least one photoinitiator is selected from the group consisting of eosin Y, 2-hydroxy-2-methylpropiophenone, 2-methyl-4′-(methylthio)-2-morpholinopropiophenone, lithium phenyl-2,4,6-trimethylbenzoylphosphinate (LAP), and 2-hydroxy-4′-(2-hydroxyethoxy)-2-methylpropiophenone (Irgacure).

In certain embodiments, the composition further comprises at least one additional compound selected from the group consisting of triethanolamine (TEOA), and N-vinylcaprolactam (VC).

In certain embodiments, the composition further comprises a biocompatible aqueous buffer.

In certain embodiments, the composition comprises about 10% to about 20% (w/v) choline acrylate.

In certain embodiments, the composition comprises about 10% to about 30% (w/v) polymer.

In certain embodiments, the composition comprises about 0.1 mM eosin Y.

In certain embodiments, the composition comprises 0.5% (w/v) LAP.

In certain embodiments, the composition further comprises about 1.5% (w/v) TEOA.

In certain embodiments, the composition further comprises about 1% (w/v) VC.

In certain embodiments, the polymerized composition forms a seal on the wound. In certain embodiments, the polymerized composition forms a hemostatic seal. In certain embodiments, the polymerized composition forms an air tight seal onto the wound.

In certain embodiments, the polymerized composition forms a seal having a burst pressure of at least 5 kPa. In certain embodiments, the polymerized composition forms a seal having an adhesion strength of at least 100 kPa. In certain embodiments, the polymerized composition forms a seal having a shear strength of at least 500 kPa.

In certain embodiments, the polymerized composition forms an antimicrobial seal on the wound. In certain embodiments, the polymerized composition forms an antibacterial seal on the wound.

In certain embodiments, the polymerized composition forms a seal capable of inhibiting the growth, proliferation and/or survival of at least one bacterial strain selected from the group consisting of Aggregatibacter actinomycetemcomitans, Porphyromonas gingivalis, Prevotella intermedia, Bacteroides forsythus, Campylobacter rectus, Eubacterium nodatum, Peptostreptococcus micros, Staphylococcus intermedius, Pseudomonas aeruginosa, Acinetobacter baumannii and Treponema sp.

In certain embodiments, the polymerized composition retains a seal on the wound for at least 28 days.

In certain embodiments, the method increases the rate of clotting in the wound. In certain embodiments, the wound is in an organ selected from the group consisting of a liver, a lung, a heart, a stomach, an intestine, a pancreas, a kidney, a bladder, an artery, a vein, a skin, a brain, and a joint tissue.

In certain embodiments, the composition is contacted with the wound during a surgical procedure. In certain embodiments, the composition is contacted with the wound via injection.

In certain embodiments, the method further comprises suturing the wound.

In certain embodiments, the method does not further comprise suturing the wound.

In certain embodiments, the subject is a mammal.

In certain embodiments, the mammal is a human.

### BRIEF DESCRIPTION OF THE FIGURES

For the purpose of illustrating the invention, depicted in the drawings are certain embodiments of the invention. However, the invention is not limited to the precise arrangements and instrumentalities of the embodiments depicted in the drawings.

FIGS. 1A-1I illustrate synthesis and characterization of choline functionalized gelatin methacrylate hydrogels and poly(ethylene glycol) diacrylate (PEGDA)polymer. FIG. 1A comprises a scheme of the acrylation of choline bicarbonate or chloline bitartarate to form acrylated choline (BIL). FIG. 1B comprises a FTIR graph showing acrylation of the choline bitartrate indicated at the peak 1700 and 3200 cm-1. FIG. 1C comprises a ¹H-NMR analysis of choline acrylate. FIG. 1D comprises a scheme of the reaction between gelatin methacrylate (GelMA) or poly(ethylene glycol) diacrylate (PEGDA)polymer and BIL to form BioGel or BioPEG respectively. FIG. 1E comprises a ¹H-NMR analysis of BIL. FIG. 1F comprises a ¹H-NMR analysis of GelMA. FIG. 1G comprises a ¹H-NMR analysis of GelMA/BIL composite hydrogel. GelMA/BIL hydrogels were formed by using 1% VC, 1.5% TEOA, and 0.1 mM Eosin Y at 120 s light exposure. FIG. 1H comprises a ¹H-NMR analysis of BioGel adhesives formed by using 0.5% Lithium phenyl-2,4,6-trimethylbenzoylphosphinate (LAP) at 60 s light exposure. FIG. 1H comprises a ¹H-NMR analysis of BioPEG adhesives formed by using 0.5% Lithium phenyl-2,4,6-trimethylbenzoylphosphinate (LAP) at 60 s light exposure.

FIGS. 2A-2E comprise images and graphs showing mechanical and physical characterization of GelMA/BIL hydrogels. FIG. 2A comprises a set of representative SEM images of GelMA/BIL hydrogels formed using 15% (w/v) GelMA with 0% (w/v) BIL (left image) and 15% (w/v) BIL (right image). FIG. 2B comprises a graph reporting swelling ratios of GelMA/BIL hydrogels at 15% (w/v) GelMA concentration with varying amounts of BIL. FIG. 2C comprises a graph showing average pore sizes for GelMA/BIL hydrogels at 15% (w/v) GelMA concentration with varying amounts of BIL. FIG. 2D comprises a graph showing degradation of GelMA/BIL hydrogels at 15% (w/v) GelMA concentration with varying amounts of BIL over the course of 14 days. FIG. 2E comprises a graph showing the effect of BIL concentration on compressive modulus of GelMA/BIL hydrogels at 25% (w/v). Error bars indicate standard error of the means, asterisk marks signify levels of p<0.05 (*), p<0.01 (**), and p<0.001 (***). Hydrogels were formed with 1 (% w/v) VC, 1.5 (% w/v) TEOA, and 0.1 mM Eosin Y at 120 s light exposure.

FIGS. 3A-3H comprise images and graphs showing in vitro 3D encapsulation of C2C12 muscle cells in GelMA/BIL hydrogels. FIGS. 3A-3C comprise images taken from live/dead assays at 7, 10 and 14 days respectively. FIGS. 3D-3F comprise images taken from F-actin/DAPI assays at 7, 10 and 14 days respectively. GelMA/BIL hydrogel with 10% (w/v) GelMA and 5% (w/v) BIL was used to perform the in vitro study. FIG. 3G comprises a graph quantifying metabolic activity as measured in relative fluorescence units (RFU), using a Presto/Blue assay at days 1, 4, 7, 10 and 14 post-encapsulation. FIG. 3H comprises a graph quantifying cell proliferation based on DAPI stained cell nuclei at days 1, 4, 7, 10 and 14 post-encapsulation.

FIG. 4 comprises a graph showing the effect of varying BIL concentrations on the adhesion properties of the resulting hydrogels. Using a hydrogel with 25% (w/v) GelMA, BIL concentration was altered and a lap shear test was conducted to determine shear strength of the sealants in comparison with commercial sealants COSEAL® and EVICEL®. Curing time: 2 min, TEOA: 1.5 (% w/v), VC:1 (% w/v), 0.1 mM Eosin Y, *p<0.05; **p<0.01; ***p<0.001; ****.

FIGS. 5A-5G comprise images and graphs showing the effect of varied BIL functionalization on antimicrobial properties of GelMA hydrogels. FIGS. 5A-5B comprise images of an agar based assay against Pseudomonas and Staphylococcus bacteria strains. As can be seen in the images, the bacterial inhibition halo grows larger as a function of increased BIL functionalization. FIG. 5C comprise a graph quantifying bacterial growth inhibition plotted against the extent of BIL functionalization of the GelMA polymers. FIGS. 5D and 5F comprise images of CFU assays against Staphylococcus and Pseudomonas bacterial strains. As can be seen in the images, the bacterial growth inhibition becomes more pronounced as a function of increased BIL functionalization. FIGS. 5E and 5G comprise graphs quantifying bacterial growth inhibition plotted against the extent of BIL functionalization of the GelMA polymers. One-way Anova *p<0.01; ***p<0.001 FIGS. 6A-6B comprise images and a graph showing the effect of BIL functionalization on the rate of whole blood clotting. FIG. 6A illustrates a 56-well plate containing various formulations of GelMA/BIL hydrogels and whole blood samples, showing that as BIL functionalization increased, clotting time decreased. FIG. 6B comprises a graph quantifying the results shown in FIG. 6A. Control wells only contain whole blood and saline solution. One-way ANOVA *p<0.01; ***p<0.001.

FIGS. 7A-7E comprise graphs and images showing in vivo biodegradation and biocompatibility of GelMA/BIL hydrogels in a rat subcutaneous model. FIGS. 7A-7B comprise a graph and images evaluating in vivo biodegradation of GelMA/BIL hydrogels on days 0, 4, 14 and 28 post implantation (n=4). FIGS. 7C-7E illustrate hematoxylin and eosin (H&E) staining of GelMA/BIL hydrogel sections 4 (FIG. 7C), 14 (FIG. 7D) and 28 (FIG. 7E) days post implantation (scale bars=500 μm).

FIGS. 8A-8E comprise images and graphs showing experiments testing the in vivo sealing capacity of GelMA sealants using a rat lung incision model. FIGS. 8A-8C comprise images and a scheme showing the lung incision and sealing procedure wherein GelMA sealant was applied to a lung leakage via a small lateral thoracotomy and UV crosslinked until the incision was sealed. FIG. 8D comprises a graph reporting burst pressure of GelMA-sealed, EVICEL® sealed, PROGEL™ sealed and suture sealed lungs immediately after material application. FIG. 8E comprises a graph reporting burst pressure of GelMA-sealed lungs on day 0 and day 7 post surgery compared to healthy lungs (**p<0.01; ***p<0.001).

FIGS. 9A-9E comprise images and a graph showing experiments testing in vivo sealing capacity of GelMA sealants using a porcine lung incision model. FIGS. 9A-9C comprise images showing a right lung lobe exposed via a small lateral thoracotomy wherein a standardized defect was created (dotted lines in FIGS. 9A-9B) and then sealed by photocrosslinking of GelMA sealant (dotted line in FIG. 9C). FIGS. 9D-9E illustrate the results of ultrasound studies on the sealed lung tissue at postoperative days 7 and 14. Freedom from pneumothorax was confirmed by sonography, as shown in the representative ultrasound image at day 14 (FIG. 9E).

FIGS. 10A-10F comprise graphs showing in vitro sealing properties of the BioGel and BioPEG. FIGS. 10A-10B illustrate graphs for standard lap shear test to determine the shear strength of the sealants (n≥5) with different percentages of Bio Ionic liquid (BIL) concentration. FIGS. 10C-10D comprise graphs for standard wound closure using porcine skin as the biological substrate to test the adhesion strength of the sealant (n≥5) with different percentages of Bioionic liquid (BIL) concentration. FIGS. 10E-10F comprise graphs for standard burst pressure test to evaluate the burst pressure of the sealant (n≥5) with different percentages of Bioionic liquid (BIL). Data are means±SD. P values were determined by one-way ANOVA followed by Tukey's multiple comparisons test (*P<0.05, **P<0.01, ***P<0.001).

FIGS. 11A-11F illustrate in vitro clotting assay of BioGel and BioPEG. FIGS. 11A-11B comprise digital pictures of the well plate of clotting assay with increasing concentration of BIL. SEM of the coagulation of RBC with control [25 (w/v) % GelMA] and BioGel [25 (w/v) % GelMA, 20 (w/v) % BIL] (FIG. 11C) and control [25 (w/v) % PEGDA] and BioPEG [25 (w/v) % PEGDA, 20 (w/v) % BIL] (FIG. 11D). FIGS. 11E-11F comprise graphs showing decrease in clotting time with increasing concentration of Bio ionic liquid. Data are means±SD. P values were determined by one-way ANOVA followed by Tukey's multiple comparisons test (*P<0.05, **P<0.01, ***P<0.001).

FIGS. 12A-12E illustrate in vitro and in vivo compatibility of the polymer-IL composites. FIG. 12A illustrates quantification of metabolic activity, relative fluorescence units (RFU), using PrestoBlue assay. FIG. 12B illustrates quantification of cell viability of live/dead images. FIG. 12C illustrates quantification of cell proliferation based on DAPI-stained cell nuclei. FIGS. 12D-12E illustrate hematoxylin and eosin (H&E) staining and Fluorescent immunohistochemical analysis, macrophage (CD68) of BioGel and BioPEG and surrounding tissue after (1) 4, (ii) 14, and (iii) 28 days of implantation, counterstained with nuclei (DAPI). Data are means±SD. P values were determined by one-way ANOVA followed by Tukey's multiple comparisons test (*P<0.05, **P<0.01, ***P<0.001).

FIGS. 13A-13I illustrate ex vivo performance characterization of the polymer-IL composites. FIGS. 13A-13C illustrate puncture, sealing and patching of the wound, respectively, in porcine heart. FIGS. 13D-13E illustrate burst pressure measurement in explanted heart and lung comparing GelMA and PEGDA with BioGel and BioPEG. FIGS. 13F-13G illustrate an in vivo tail cut model to estimate the loss in % total blood volume.

FIGS. 13H-13I illustrate a liver laceration model to estimate the % TBV comparing GelMA and PEGDA with BioGel and BioPEG. Data are means±SD. P values were determined by one-way ANOVA (*P<0.05, **P<0.01, ***P<0.001).

FIGS. 14A-14H comprise graphs showing in vitro swelling, degradation and mechanical characterization of the BioGel [25% (w/v) GelMA with varying concentration of BIL] and BioPEG [25% (w/v) PEGDA with varying concentration of BIL] synthesis by photopolymerization under visible light using 0.5% LAP as photoinitiator. BioGel degradation profiles in DPBS over a two-weeks period are illustrated in FIG. 14A and swelling ratio in DPBS after 1, 2, 4, 6, 8 and 24 h are illustrated in FIG. 14B. Mechanical characterization of BioGel compression is shown in FIG. 14C and elastic modulus is illustrated in FIG. 14D. BioPEG degradation profiles in DPBS over a two-weeks period are illustrated in FIG. 14E and swelling ratio in DPBS after 1, 2, 4, 6, 8 and 24 h are illustrated in FIG. 14F. Mechanical characterization of BioPEG compression is illustrated in FIG. 14G and elastic modulus is illustrated in FIG. 14F. Data are means±SD. P values were determined by one-way ANOVA followed by Tukey's multiple comparisons test (*P<0.05, **P<0.01, ***P<0.001).

FIG. 15 illustrates in vitro sealing properties of the BioGel. Standard lap shear test to determine the shear strength of the sealants (n≥5) with different percentages of GelMA and Bio Ionic liquid (BIL) concentration (0-20 (w/v)] % concentration. Data are means±SD. P values were determined by one-way ANOVA followed by Tukey's multiple comparisons test (*P<0.05, **P<0.01, ***P<0.001).

FIGS. 16A-16B illustrate in vitro sealing properties of the BioGel and BioPEG sealant compared to commercially available sealants: Evicel, Coseal, and Progel. FIG. 16A illustrates standard lap shear test, and FIG. 16B illustrates standard burst pressure test. The data for the commercially available sealant are reproduced from references. Data are mean±SD. P values were determined by one-way ANOVA followed by Tukey's multiple comparisons test (*P<0.05, **P<0.01, ***P<0.0001).

FIGS. 17A-17D illustrate in vitro biocompatibilty of BioGel and BioPEG. Representative live/dead images and F-Actin/DAPI fluorescent images at days 1, 4 and 7 post seeding of BioGel (FIGS. 17A-17B) and BioPEG (FIGS. 17C-17D).

### DETAILED DESCRIPTION OF THE INVENTION

The present invention relates to the discovery of methods of treating a wound in a subject in need thereof. In certain embodiments, the method comprises contacting the wound with a composition comprising a polymer such as, but not limited to, gelatin methacrylate (GelMa) or poly(ethylene glycol) diacrylate (PEGDA), wherein the composition further comprises choline acrylate, and then polymerizing the composition to form a polymerized composition having a plurality of choline acrylate functionalized polymer units. The following is a non-limiting illustration of a composition of the invention.

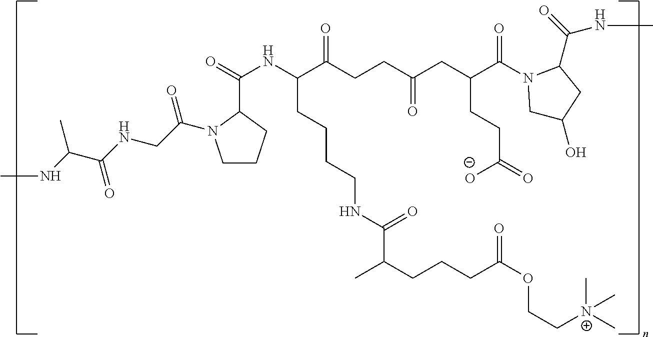

### DISCLOSURE

Ionic liquids (ILs) are organic salts with a low melting point and high water solubility. ILs have emerged as promising alternatives in the field of material synthesis, due to their high thermal stability, conductivity, antimicrobial and antifouling properties. Among the variety of ILs, choline-based Bio-Ionic Liquid (BILs) have gained much interest due to their enhanced biocompatibility. Choline is a precursor of the phospholipids that comprise biological cell membranes in mammalian and plant tissues, such as phosphatidylcholine and sphingomyelin. Previous studies have shown that choline can be decomposed both physiologically and environmentally into smaller chain molecules. Consequently, choline-based BILs have been investigated as non-toxic components for numerous applications. Unlike conventional ILs, BILs are biodegradable and non-cytotoxic, as they are comprised solely of naturally derived compounds.

### Methods

In one aspect the invention provides methods of treating a wound in a subject in need thereof using choline acrylate functionalized gelatin methacrylate polymer (BioGel) or choline acrylate functionalized poly(ethylene glycol) diacrylate polymer (BioPEG).

In certain embodiments, the method comprises contacting the wound with a composition comprising choline acrylate, gelatin methacrylate and at least one photoinitiator. In certain embodiments, the method comprises exposing the composition to at least one wavelength of light capable of activating the at least one photoinitiator, thereby polymerizing the composition.

In certain embodiments, the composition comprises about 1:4 to about 4:1 choline acrylate to gelatin methacrylate. In other embodiments, the composition comprises about 1:1 choline acrylate to gelatin methacrylate.

In certain embodiments, the composition comprises about 1:4 to about 4:1 choline acrylate to poly(ethylene glycol diacrylate). In other embodiments, the composition comprises about 1:1 choline acrylate to poly(ethylene glycol diacrylate).

In certain embodiments, the at least one photoinitiator is reactive upon exposure to light in the IR (700-1,000,000 nm), visible (400-700 nm) or UV (10-400 nm). In other embodiments, the at least one photoinitiator is selected from the group consisting of eosin Y, 2-hydroxy-2-methylpropiophenone, 2-methyl-4′-(methylthio)-2-morpholinopropiophenone, lithium phenyl-2,4,6-trimethylbenzoylphosphinate (LAP), and 2-hydroxy-4′-(2-hydroxyethoxy)-2-methylpropiophenone (Irgacure).

In certain embodiments, the composition further comprises at least one additional compound selected from the group consisting of triethanolamine (TEOA) and N-vinylcaprolactam (VC).

In certain embodiments, the method seals the wound at least partially. As used herein, the term “seal” refers to a physical barrier between the wound and the surrounding tissue, organ, and/or environment (including, for examples, air or a bodily fluid). In certain embodiments, the seal is a solid or semi-solid film formed on the surface of the wound. In other embodiments, the seal reduces. minimizes, and/or avoids contact between the wound and the environment around the wound, including air, fluids, or at least a portion of microorganisms therein.

In certain embodiments, the method provides a hemostatic seal. In other embodiments, the method provides an air tight seal.

In certain embodiments, the polymerized composition forms a seal having a burst pressure of at least 5 kPa. In other embodiments, the polymerized composition forms a seal having an adhesion strength of at least 100 kPa. In yet other embodiments, the polymerized composition forms a seal having a shear strength of at least 500 kPa.

In certain embodiments, the method provides an antimicrobial seal on the wound. In other embodiments, the method provides an antibacterial seal on the wound. In yet other embodiments, the method provides a seal capable of inhibiting the growth, proliferation and/or survival of at least one bacterial strain selected from the group consisting of Aggregatibacter actinomycetemcomitans, Porphyromonas gingivalis, Prevotella intermedia, Bacteroides forsythus, Campylobacter rectus, Eubacterium nodatum, Peptostreptococcus micros, Staphylococcus intermedius, Pseudomonas aeruginosa, Acinetobacter baumannii, and Treponema sp.

In certain embodiments, the method increases the rate of blood clotting in the wound. In certain embodiments, the method reduces clotting time in the wound by up to 95%.

In certain embodiments, the polymerized composition is kept in contact with the wound until the wound heals. In other embodiments, the polymerized composition is biodegradable and degrades over time within the body of the subject. In certain embodiments, the polymerized composition retains a seal on the wound for at least 28 days before degrading.

In certain embodiments, the composition, the polymerized composition, and the metabolic degradation products of the polymerized composition are non-toxic to the subject.

In certain embodiments, the method does not induce significant toxicity in the subject. In other embodiments, the method does not induce a significant allergic reaction in the subject. In yet other embodiments, the method does not induce a significant inflammatory response in the subject.

In certain embodiments, the wound is in an organ selected from the group consisting of a liver, a lung, a heart, a stomach, an intestine, a pancreas, a kidney, a bladder, an artery, a vein, a skin, a brain, and a joint tissue (eg. a knee, an elbow, and so forth).

In certain embodiments, the composition further comprises a solvent. In other embodiments, the solvent comprises water. In yet other embodiments, the solvent is a biocompatible aqueous solution, such as but not limited to a phosphate buffered saline (PBS). In certain embodiments, the composition comprises about 5% to about 50% (w/v) choline acrylate. In other embodiments, the composition comprises about 10% to about 20% (w/v) choline acrylate. In yet other embodiments, the composition comprises about 15% (w/v) choline acrylate. In certain embodiments, the composition comprises about 5% to about 50% (w/v) gelatin methacrylate. In other embodiments, the composition comprises about 10% to about 30% (w/v) gelatin methacrylate. In other embodiments, the composition comprises about 25% (w/v) gelatin methacrylate. In yet other embodiments, the composition comprises about 0.1 mM eosin Y. In yet other embodiments, the composition comprises 0.5% (w/v) LAP. In yet other embodiments, the composition further comprises about 1.5% (w/v) TEOA. In yet other embodiments, the composition further comprises about 1% (w/v) VC.

In certain embodiments, the composition is contacted to the wound during a surgical procedure. In other embodiments, the composition is contacted to the wound via injection using a syringe.

In certain embodiments, the method further comprises suturing the wound. In other embodiments, the method does not further comprise suturing the wound.

In certain embodiments, the composition is exposed to the at least one wavelength of light through exposure to at least one light source. In other embodiments, the at least one light source is selected from the group consisting of a light bulb, a light emitting diode (LED), and a fiber optic cable.

In certain embodiments, the choline acrylate is synthesized by reacting acrylic acid with at least on choline salt selected from the group consisting of choline bicarbonate and choline bitartrate.

In certain embodiments, the subject is a mammal. In other embodiments, the subject is a human.

### Definitions

As used herein, each of the following terms has the meaning associated with it in this section.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

Generally, the nomenclature used herein and the laboratory procedures in tissue engineering and biomaterial science are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

A “bio-ionic liquid” as used herein refers to a salt that has a melting temperature below room temperature (e.g., the melting temperature is less than 10° C., less than 15° C., less than 20° C., less than 25° C., less than 30° C., or less than 35° C.) and that contains a cation and an anion, at least one of which is a biomolecule (i.e., a molecule found in a living organism) or a biocompatible organic molecule. Examples of bio-ionic liquids are organic salts of choline, such as carboxylate salts of choline, choline bicarbonate, choline maleate, choline succinate, and choline propionate. An ionic constituent of a bio-ionic liquid is a cation or anion component of a bio-ionic liquid. Examples of ionic constituents of bio-ionic liquids for use in the invention are biocompatible organic cations such as choline and other biocompatible quaternary organic amines, as well as biocompatible organic anions such as carboxylic acids, including formate, acetate, propionate, butyrate, malate, succinate, citrate, and the like.

A “biocompatible polymer” as used herein refers to an organic polymer found in a living organism or compatible with a living organism. The polymer can be naturally occurring or synthetic and charged or uncharged. The polymer is sufficiently hydrophilic to be capable of forming a hydrogel or serving as a component of a hydrogel. Examples of biocompatible polymers for use in the invention include gelatin, elastin, elastin like polypeptides (ELP), chitosan, tropoelastin, collagen, hyaluronic acid (HA), alginate, poly(glycerol sebacate) (PGS), poly(ethylene glycol) (PEG), and poly(lactic acid) (PLA). A biocompatible polymer, conjugate, or other molecule or composition is capable of being in contact with cells without compromising their viability, such as by causing cell death, inhibition of cell proliferation, or exhibiting toxic effects on cellular metabolism or physiology of the organism. For example, a hydrogel is biocompatible if cells applied on its surface or embedded within its matrix remain viable as measured over a period of days, e.g., 5 days, 10 days, or 30 days.

As used herein, the terms “functionalized”, “covalently bound” or “covalently conjugated” refers to the formation of a covalent bond between two chemical species or moieties. Covalent bonds are to be taken to have the meaning commonly accepted in the art, referring to a chemical bond that involves the sharing of electron pairs between atoms.

As used herein, the term “gel” refers to a three-dimensional polymeric structure that itself is insoluble in a particular liquid but which is capable of absorbing and retaining large quantities of the liquid to form a stable, often soft and pliable, but always to one degree or another shape-retentive, structure. When the liquid is water, the gel is referred to as a hydrogel. Unless expressly stated otherwise, the term “gel” is used throughout this application to refer both to polymeric structures that have absorbed a liquid other than water and to polymeric structures that have absorbed water, it being readily apparent to those skilled in the art from the context whether the polymeric structure is simply a “gel” or a “hydrogel.”

The terms “patient,” “subject” or “individual” are used interchangeably herein, and refer to any animal, or cells thereof whether in vitro or in situ, amenable to the methods described herein. In a non-limiting embodiment, the patient, subject or individual is a human. In other embodiments, the subject is a non-human mammal including, for example, livestock and pets, such as ovine, bovine, porcine, canine, feline, primate and murine mammals.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. The following abbreviations are used herein: BIL=bio-ionic liquid (more specifically, choline acrylate), CFU=Colony forming units, DPBS=Dulbecco's phosphate buffered saline, DSC=Differential Scanning calorimetry, FTIR=Fourier transform infrared spectroscopy, GelMA=gelatin methacrylate, GelMA/BIL or BioGel=choline acrylate functionalized gelatin methacrylate, PEGDA=poly(ethylene glycol) diacrylate, PEGDA/BIL or BioPEG=choline acrylate functionalized poly(ethylene glycol) diacrylate, GPC=Gel Permeation Chromatography, MA=methacrylic acid, LAP=lithium phenyl-2,4,6-trimethylbenzoylphosphinate, MIC=minimum inhibitory concentration, MHB=Mueller Hinton Broth, OCT=Optimal cutting temperature compound, OD=Optical density, PDMS=polydimethylsiloxane, RBCs=red blood cells, TEOA=triethanolamine, Tg=glass transition temperature, VC=N-vinylcaprolactam.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination. Although the description herein contains many embodiments, these should not be construed as limiting the scope of the invention but as merely providing illustrations of some of the presently preferred embodiments of the invention.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application. In general the terms and phrases used herein have their art-recognized meaning, which can be found by reference to standard texts, journal references and contexts known to those skilled in the art. Any preceding definitions are provided to clarify their specific use in the context of the invention.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### Synthesis of Choline Acrylate (BIL)

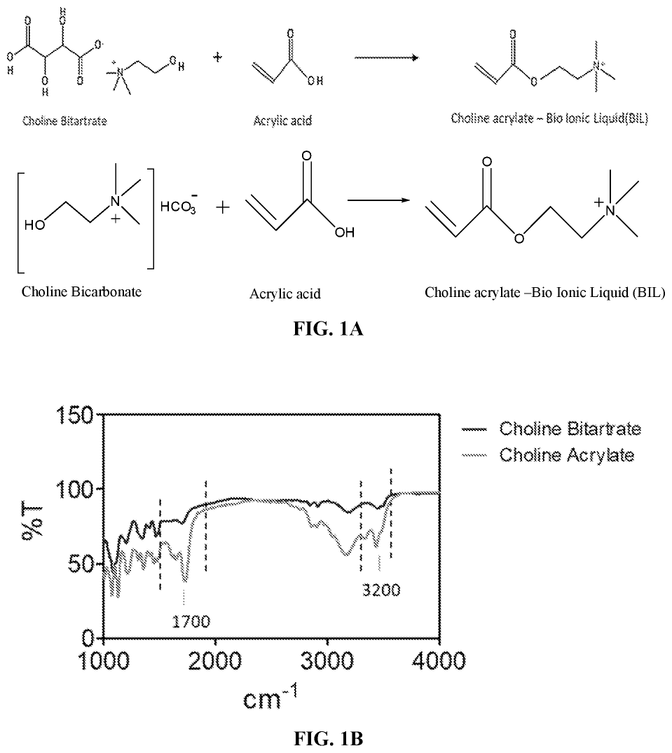

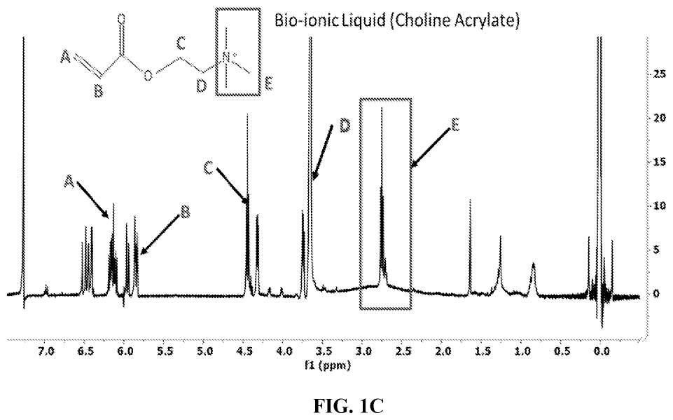

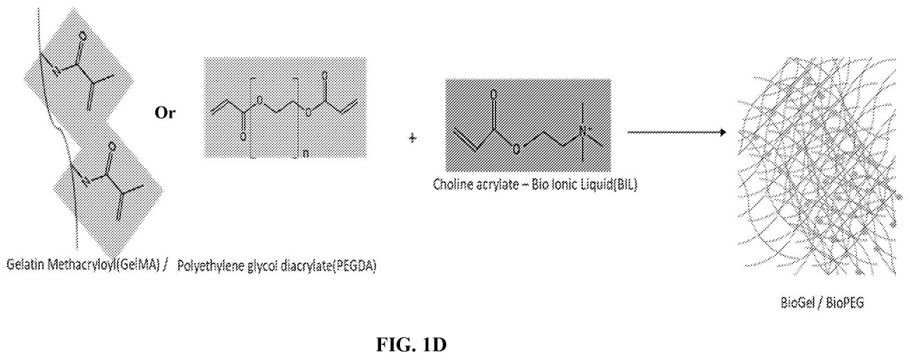

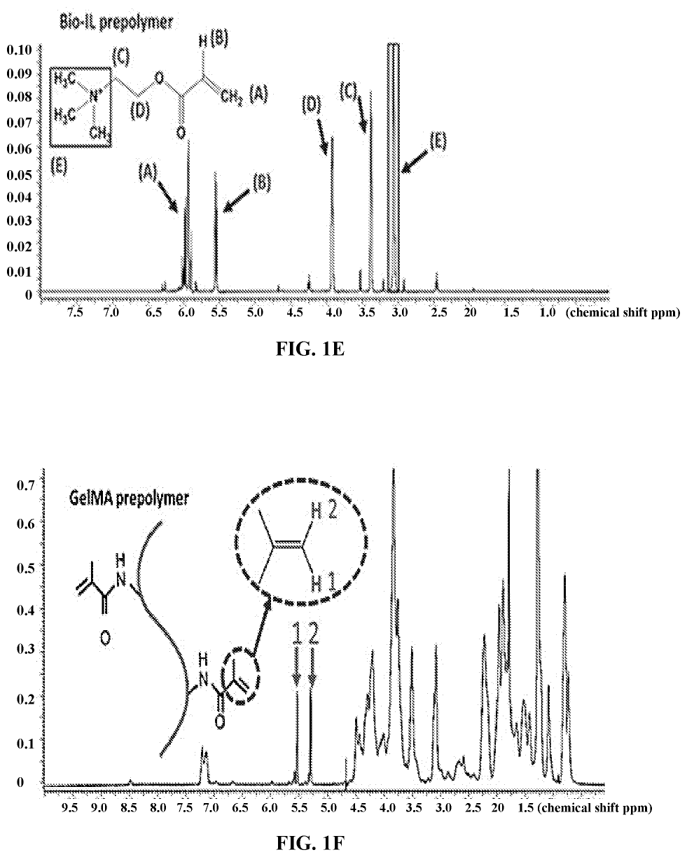

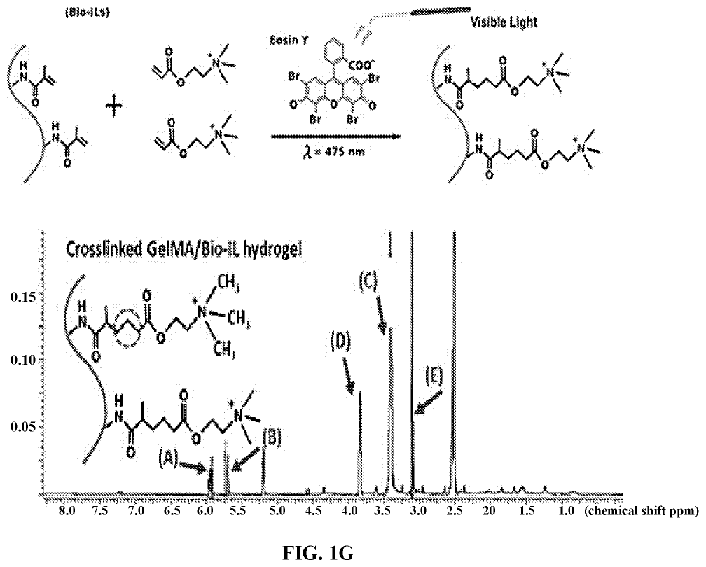

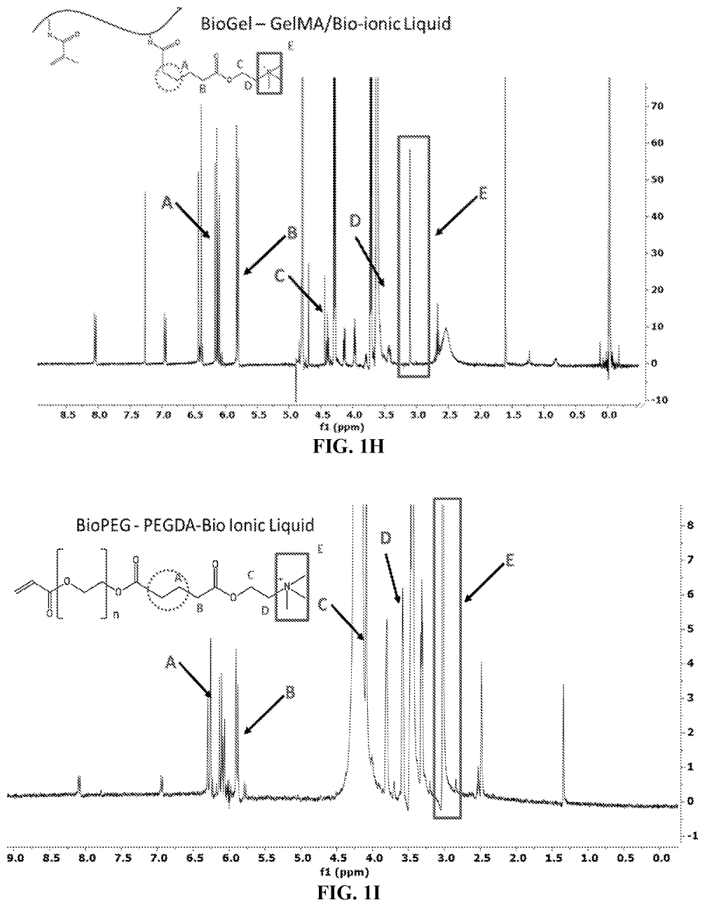

Choline bicarbonate 1 mol or Choline bitartrate (1 mol) was mixed with acrylic acid (1 mol) and reacted at 50° C. for 5 hr under an inert nitrogen atmosphere. The remaining acrylic acid was removed from the reaction mixture by extracting with methylene chloride (CH₂Cl₂). The acrylated BIL was purified with rotary evaporation for 24 hr followed by freeze drying. (FIGS. 1A, 1D).

The progress of the acylation of choline bitartrate to make choline acrylate was tracked using FTIR (FIG. 1B) and NMR (FIG. 1C). In the FTIR figure, the appearance of a peak at 1720 cm⁻¹ indicates the formation of the ester bond via the acrylation. Similarly, in NMR, the peak related to the hydrogen atom of acrylate in the NMR at 5.9-6.3 ppm indicates the acrylation of the choline bitartrate forming choline acrylate.

### Synthesis of Gelatin Methacrylate

Type A porcine skin gelatin was mixed at 10% (w/v) into Dulbecco's phosphate buffered saline (DPBS; GIBCO) at 60° C. and stirred until fully dissolved. MA was added until the target volume was reached at a rate of 0.5 mL/min to the gelatin solution under stirred conditions at 50° C. and allowed to react for 1 h. The fraction of lysine groups reacted was modified by varying the amount of MA present in the initial reaction mixture. Following a 5× dilution with additional warm (40° C.) DPBS to stop the reaction, the mixture was dialyzed against distilled water using 12-14 kDa cutoff dialysis tubing for 1 week at 40° C. to remove salts and methacrylic acid. The solution was lyophilized for 1 week to generate a white porous foam and stored at −80° C. until further use (FIG. 1F).

Alternatively, GelMA was synthesized using the following method. Briefly, 10% (w/v) gelatin solution was reacted with 8 mL of methacrylic anhydride for 3 h under inert conditions. The solution was then dialyzed for 5 days to remove any unreacted methacrylic anhydride, and then placed in a −80° C. freezer for 24 h. The frozen acrylated polymer was then freeze-dried for 7 days.

Poly(Ethylene Glycol) Diacrylate (PEGDA) The PEGDA used for this research was purchased from Sigma Aldrich (average Mₙ 700).

### General Route for BioGel and BioPEG Synthesis

GelMA and PEGDA were the two polymers used to synthesize BioGel and BioPEG adhesives, respectively, along with BIL. Different ratios of methacrylated polymers and choline acrylate (bio-ionic liquid) were mixed together to form the adhesive, the prepolymer and ionic liquid were added to distilled water at varying final polymer concentrations and polymer/BILs ratios and mixed with 0.5% (w/v) LAP which acted as the photo-initiator. Hydrogels were then applied to the requisite surfaces and rapidly photo-crosslinked in the presence of visible light at a wavelength of 450 nm for 120 s and 60 s for GelMA and PEGDA, respectively.

### Conjugation of Acrylated Choline to Gelatin Methacrylate (GelMA) and Compression Tests

Seventy microliters of a prepolymer mixture containing gelatin methacrylate and acrylated choline (GelMA/BIL) in triethanolamine (TEOA) and N-vinylcaprolactam (VC) were injected into polydimethylsiloxane (PDMS) molds and exposed to visible light (450 nm) for 120 seconds. The PDMS material contained rectangular (w: 5 mm, l: 12 mm, d: 1.25 mm) and cylinder-shaped molds (d: 5.5 mm, h: 4 mm) for conducting tensile and compression tests, respectively. Samples were removed from the molds and placed in DPBS for 2 hours at room temperature. Hydrogels were blotted dry and measurements for swelling were made using digital calipers before positioning them in an Instron 5542 mechanical tester with a 10 N load cell. Compression was performed at 1 mm/min of speed until failure occurred. Compression modulus was calculated as the slope of the initial linear region at the stress-strain curve obtained by plotting the results of compressions. Conjugation of the acrylated choline to the GelMA was confirmed by ¹H-NMR (FIG. 1G).

### NMR Analysis

¹H NMR analyses were performed to characterize polymer/ionic liquid composite hydrogels using a Varian Inova-500 NMR spectrometer. ¹H-NMR spectra of choline bitartrate, choline acrylate, GelMA prepolymer, and adhesive samples were obtained. The decreasing rate for the C═C double bond signals (−∂(C═C)∂t) in methacrylate group of GelMA was associated with the extent of crosslinking of composite hydrogel as well as conjugation of ionic liquid to polymer. This area decrease was calculated using the following equation:

Decay of Methyl group %=[(PA_{b}−PAₐ)/PA_{b}]*100

where PA_{b} and PAₐ represent the peak areas of methacrylated groups before and after photocrosslinking, respectively. Accordingly, PA_{b}−PAₐ correspond to the concentration of methacrylated groups consumed in the photocrosslinking process.

### Mechanical Testing

Mechanical testing on adhesives were performed by using an Instron Universal Testing Machine. Both elastic and compressive modulus were analyzed for each of the hydrogels and the moduli analyzed vis-a-vis various levels of ionic liquid loading in the polymer. Composite hydrogels were created using PDMS molds of cylindrical dimensions—diameter: 5.5 mm, height: 4 mm—for mechanical tests. These were allowed to swell in DPBS for 4 h at 37° C. prior to commencement of mechanical testing. At least 5 samples were tested for each condition.

For the compression tests, the hydrogels were placed between two compression plates, and compressive stress was applied to each sample at a rate of 1 mm/min. The compression (mm) and load (N) were recorded during each test using. Compression modulus was calculated as the tangent slope of the initial linear region of the stress-strain curve between 0 mm/mm and 0.1 mm/mm compressive strain. For the tensile tests, the hydrogels were held between two tensile grips and stretched at a rate of 1 mm/min until failure. The elastic modulus was calculated as the tangent slope of the stress-strain curve. At least 5 samples were tested per condition to obtain average and standard deviation.

### In Vitro Degradation Tests

BioGel and BioPEG were fabricated as previously explained for compression test. The BioGel and BioPEG samples were then freeze-dried, weighed and were placed in 24-well plate with 1 ml of DPBS or at 37° C. in an oven continuously for 2 weeks. The DPBS/FBS solutions were refreshed every 3 days to maintain constant enzyme activity. At prearranged time points (after 1, 7 and 14 days), the DPBS/FBS solutions were removed and the samples were freeze-dried for 24 h and weighed. The percentage degradation (D %) of the hydrogels was calculated in terms of the loss of weight.

### Swelling Ratio Measurements

The equilibrium swelling ratio of BioGel composite hydrogels were evaluated. For this purpose, cylinder-shaped hydrogels were prepared (7 mm in diameter, 2 mm in depth) as described previously. Prepared hydrogels were washed three times with DPBS. Then, they were lyophilized and weighed in dry conditions. Thereafter, the samples were immersed in DPBS at 37° C. for 4, 8 and 24 h and weighed again after immersion. The swelling ratio and water uptake capacity of the samples were calculated as the ratio of the swollen sample mass to the mass of lyophilized sample.

### In Vitro Lap Shear

Shear strength of the BioGel and BioPEG adhesives was tested according to the modified ASTM F2255-05 standard for tissue adhesives. Two pieces of 2 cm×2 cm were cut from a glass slide. A 1 cm×1 cm layer of gelatin was pasted onto each piece of glass slide, and left to dry to function as a base layer. The remaining uncoated area was covered by a tape, which was later used to clamp the glass slides into the Instron machine. A 20 μL drop of hydrogel precursor solution was crosslinked between the two layers of gelatin coated glass slides. The two glass slides were placed in the mechanical tester, and tensile loading was applied with a strain rate of 1 mm/min. Shear strength was calculated at the point of detaching.

### In Vitro Adhesive Strength

Wound closure of the polymer/IL composite hydrogels was calculated by using the ASTM F2458-05 standard. Porcine skin was obtained from a local butcher and cut into small strips, with excess fat was removed. Tissues were immersed into PBS before testing to prevent drying in the air. The tissue was then separated in the middle with a straight edge razor to simulate the wound. 100 μL of polymer solution was administered onto the desired adhesive area and crosslinked by light. Maximum adhesive strength of each sample was obtained at the point of tearing at strain rate of 1 mm/min using a mechanical tester.

### Ex Vivo Burst Pressure

Burst pressure of polymer/IL composite was calculated by using the ASTM F2392-04 standard. Porcine heart and lung were obtained from a local butcher. A 5 mm×5 mm puncture was made in the left chamber of the heart was placed in connection from a custom built burst pressure apparatus, which consists of pressure meter, syringe pressure setup and air was flowed using a syringe pump at 0.5 ml/s. The puncture made on the intestine was covered with a crosslinked hydrogel, prior to initiating the pump and sensor. Airflow was terminated post hydrogel rupture and the burst pressure was measured.

### Clotting Time and SEM Analysis of RBC

SEM analysis was performed to evaluate the hemostatic property of the engineered adhesive, according to methods known in the art.

EDTA-anticoagulated whole blood (6 ml) was centrifuged at 2300 rpm for 5 min for preparing the RBC pellet. The plasma and buffy coat layers (platelets and white cells) were discarded. The RBC pellet was then washed with 40 ml of isotonic saline (0.9% w/v of aqueous NaCl solution, pH 7.4) and this process was repeated three times. To prepare RBC pellet, an oil mixture (1 ml, 2.6 parts by weight of benzyl benzoate and 1 part by weight of cottonseed oil) with density intermediate to RBCs and the isotonic saline was added to the washed RBCs. The combined oil mixture-RBC suspension was centrifuged at 4500 rpm for 10 min. The supernatant oil layer was discarded. The oil film over-laying the RBC pellet was removed by use of cotton-tipped applicators and incubated with the adhesive sample overnight at 37 C and dehydrated for freeze drying. The freeze-dried samples were then coated by gold/palladium (Au/Pd) before SEM analysis, and SEM images were acquired. Clotting time analysis of the adhesive was performed by measuring the coagulation of RBC of at least three images of four samples (n=50) using ImageJ software.

### Surface Seeding (2D Culture)

On the surface of hydroges, 0.05×106 cells/scaffold were seeded and placed in 24-well plates with 400 μl of growth medium (DMEM supplemented with 10% fetal bovine serum 2D cultures were maintained at 37° C. in a 5% CO2 humidified atmosphere, for 10 days and culture medium was replaced every 48 h.

### Cell Viability

The viability of primary C2C12 grown on the surface of polymer and polymer/IL adhesive was evaluated using a commercial live/dead viability kit (Invitrogen), according to instructions from the manufacturer. Briefly, cells were stained with 0.5 μl/ml of calcein AM and 2 μl/ml of ethidium homodimer-1 (EthD-1) in DPBS for 15 min at 37° C. Fluorescent image acquisition was carried out at days 1, 4, and 7 post-seeding using an Axio Observer Z1 inverted microscope (Zeiss). Viable cells appeared as green and apoptotic/dead cells appeared as red. The number of live and dead cells was quantified using the ImageJ software. Cell viability was determined as the number of live cells divided by the total number of live and dead cells.

### Metabolic Activity

The metabolic activity of the cells was evaluated at days 1, 4, 7 post-seeding, using a PrestoBlue assay (Life Technologies) according to instructions from the manufacturer. Briefly, 2D and 3D cultures of primary CMs were incubated in 400 μL of growth medium with 10% PrestoBlue reagent for 2 h at 37° C. The resulting fluorescence was measured (excitation 530 nm; emission 590 nm) using a. Control wells without cells were used to determine the background for all experiments.

### Cell Adhesion, Proliferation and Spreading

Spreading on the surface of the engineered composite hydrogels was visualized through fluorescent staining of F-actin filaments and cell nuclei. Briefly, 2D cultures at days 1, 4, and 7 post-seeding were fixed in samples were then incubated with Alexa-fluor 488-labeled rhodamine-phalloidin (20/800 dilution in 0.1% BSA, Invitrogen) for 45 min. After three consecutive washes with DPBS, samples were counterstained with 1 μl/ml DAPI (4′,6-diamidino-2-phenylindole, Sigma) in DPBS for 5 min. Fluorescent image acquisition was carried out using an AxioObserver Z1 inverted microscope.

### In Vivo Bleeding Test

Tail-Cut Model

Animals selected for the tail-cut model were positioned with the tail toward the surgeon. The tail was marked 4 cm from the tip, and transected using a scalpel. The tail stump was placed in a 1.5-mL microcentrifuge tube to collect the shed blood. Blood loss was recorded every 2 minutes for the first 10 minutes after injury and at 5-minute intervals thereafter for a total of 30 minutes.

Liver Laceration Model

Animals selected for the liver laceration model were positioned with the tail toward the surgeon. A midline laparotomy was performed to expose the abdominal cavity, and the left lobe of the liver was exposed. A 3.0×1.5-cm section of the edge of the left liver lobe was transected to cause liver injury. Pre weighed gauze was placed inferior to the liver before injury. The gauze was exchanged and weighed at intervals as previously described.

### C2C12 Cell Culture Assay

Hydrogels were formed by placing a 7 μl drop of hydrogel precursor in a spacer with 150 μm height and covered by a glass slide coated with 3-(trimethoxysilyl) propyl methacrylate (TMSPMA, Sigma-Aldrich). Hydrogel precursors were then photocrosslinked for 20 s using a Genzyme FocalSeal LS100 xenon light source. C2C12 cells (3.5×104 cells/scaffold) were seeded on the surface of the hydrogels and placed in 24-well plates with 400 μl of growth medium [DMEM supplemented with 10% fetal bovine serum (FBS, Invitrogen) and 1% penicillin/streptomycin (Invitrogen)]. 2D cultures were maintained at 37° C. in a 5% CO₂ humidified atmosphere, for 10 days and culture medium was replaced every 48 h. 3D cell encapsulation. Hydrogel precursors were prepared in cell culture medium containing 1.5% TEOA, 1% VC, and 0.1 mM Eosin Y. A cell suspension was gently mixed with an equal volume of the precursor solution. 7 μl drops were then pipetted on 150 μm thick spacers, and covered by TMSPMA-coated glass slides. Hydrogels were then photocrosslinked for 120 s using a Genzyme FocalSeal LS100 xenon light source, as described before for 2D cultures. Cell laden hydrogels were placed in 24-well plates with 400 μl of growth medium, and maintained at 37° C. in a 5% CO₂ humidified atmosphere for 10 days.

### Colony Forming Unit and Agar Plate Assay Protocols

Colony Forming Unit (CFU) were used to evaluate the antimicrobial activity of the GelMA/BIL hydrogel against Staphylococcus and Pseudomonas bacterial strains, by determining the remaining number of colony-forming units (CFU) in triplicate experiments. The bacteria samples were grown overnight in 37° C. incubators in Mueller-Hinton agar (MHA; Difco) until the colonies appeared on the MHA. Subsequently, two colonies were harvested and grown overnight in Mueller Hinton Broth (MHB; Difco) while shaking in the incubator at 37° C. To obtain bacteria in the mid-logarithmic phase of growth, 100 μL of the original bacterial solution was transferred into five mL of Mueller Hinton Broth (MHB) and incubated at 37° C. for 1 h. The bacteria suspension was diluted again with MHB to reach a final concentration of ˜10⁶ cells/mL, as determined using a spectrophotometer. Briefly, the bacteria culture was adjusted to reach OD₆₀₀ 0.3-0.5 after calibrating the spectrometer with a MHB standard sample to prepare a mid-Log (exponential phase) suspension culture. The GelMA/BIL hydrogel was placed in a 12-well culture dish, rinsed with PBS, and two mL of bacterial solution (density ˜10⁶ bacteria/mL) was added to each well. A negative control containing GelMA hydrogel was used as a basis of comparison. The samples were incubated for 1, 4 and 8 h at 37° C. in a humidified incubator with 5% CO₂, and 20% O₂, while shaking at 70 rpm. The remaining bacteria were plated on nutrient MHA agar and incubated overnight at 37° C. The surviving bacteria for each specimen was evaluated by measuring colony-forming units (CFU) by counting the number of bacteria colonies on a plate with a countable number of colonies after serially diluting the bacteria solution.

### Assay for Measuring Clotting of Whole Blood

A volume of 630 μL citrated blood was pipetted into a 1.5 mL Eppendorf tube. A total of 70 μL of 0.1 M calcium chloride (CaCl₂) was then added, followed by vortexing for 10 s. 50 μL of the resulting solution was then deposited into each sequential well on a 96 well plate. At selected time points, each well was washed with 9 g/L saline solution to halt clotting. The liquid was immediately aspirated, and repeatedly washed until the solution became clear, indicating removal of all soluble blood components.

### In Vivo Biocompatibility and Degradation Protocol

Male Wistar rats (200-250 grams) were obtained from Charles River (Boston, Mass., USA) and housed in the local animal care facility under conditions of circadian day-night rhythm and feeding ad libitum. Anesthesia was achieved by 2.0 to 2.5% isoflurane inhalation, followed by 0.02 to 0.05 mg/kg SC buprenorphine administration. After inducing anesthesia, eight 1-cm incisions were made on the posterior medio-dorsal skin, and small lateral subcutaneous pockets were prepared by blunt dissection around the incisions. GelMA/Bio-IL hydrogels (1×5 mm disks) were implanted into the pockets, followed by anatomical wound closure and recovery from anesthesia. Animals were euthanized by anesthesia/exsanguination at days 4, 14 and 28 post-implantation, after which the samples were retrieved with the associated tissue and placed in DPBS.

### Example 1: Synthesis of Choline Functionalized Gelatin Methacrylate Hydrogel (GelMA/BIL) and Choline Functionalized PEG Diacrylate (PEGDA/BIL)

### Choline Functionalized Gelatin Methacrylate Hydrogel

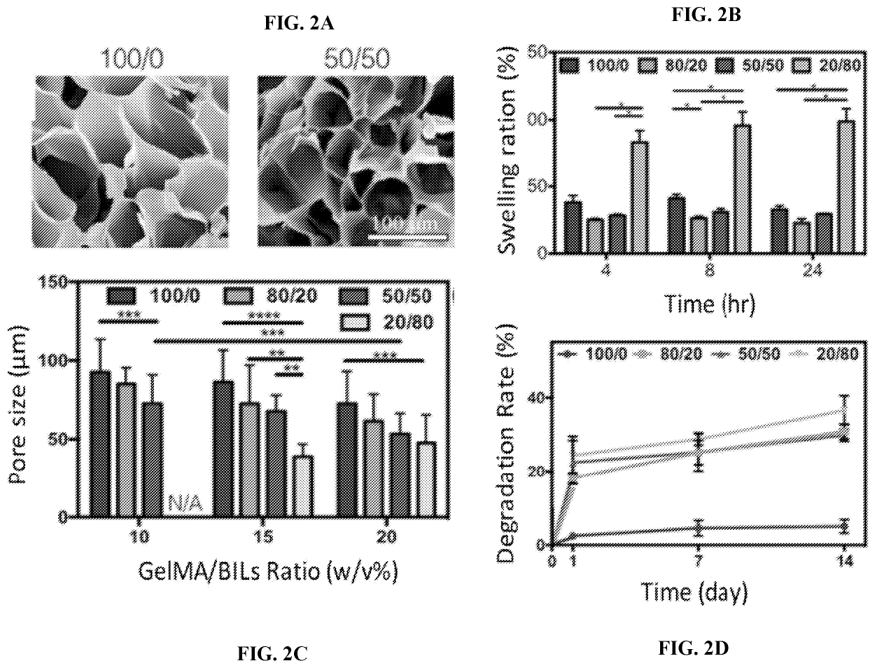

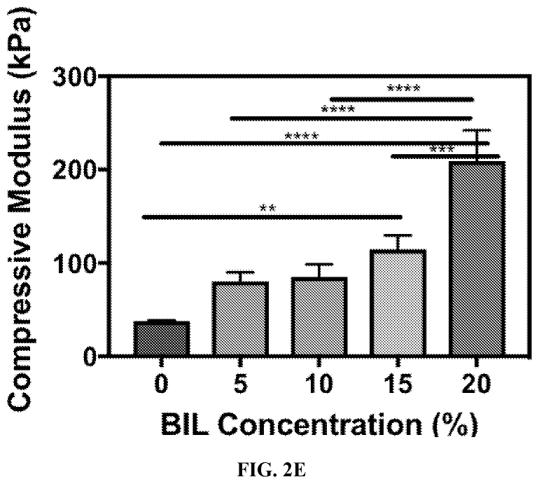

Choline functionalized gelatin methacrylate hydrogel (GelMA/BIL) was synthesized by reacting choline acrylate and gelatin methacrylate in a PBS solution triethanolamine (TEOA), eosin Y and N-vinylcaprolactam (VC) and exposing the solution with visible light to promote polymerization (FIG. 1G,). Choline functionalized gelatin methacrylate hydrogels containing various ratios of choline and gelatin methacrylate were synthesized and the mechanical and physical properties of each hydrogel were studied. It was found that the average pore size in the hydrogels was dependent on the final polymer concentration as well as the GelMA:choline ratio (FIGS. 2A-2B). Results further showed that in vitro degradation of 15% (w/v) hydrogel occurred after 24 h of incubation for all GelMA:choline ratios tested (FIG. 2D). Mechanical properties increased as a function of molecular weight (FIG. 2E)

Further optimization of the hydrogels is carried out using GelMA (85% methacrylation) prepolymer dissolved in PBS solution at concentrations ranging from 10-25% w/v, in the presence of choline acrylate at concentrations ranging from 0-20% w/v. Additionally, Eosin-Y (0.1-0.5 mM), TEOA (0.5-2% w/v), and VC (0.5-1.5% w/v) concentrations is varied as well. The formulated prepolymer solutions are photocrosslinked by exposure to commercial flashlight for 20-120 seconds, which matches the absorption spectrum of Eosin-Y. The effect of GelMA prepolymer concentration (10-25% w/v), choline acrylate concentration (0-20% w/v), Eosin-Y concentration (0.5-1.5 mM), TEOA concentration (0.5-2% w/v), and light exposure time (10-60 s) on the physical and mechanical properties of the engineered hydrogels is studied. Alternatively, LAP (Lithium phenyl-2,4,6-trimethylbenzoylphosphinate) can also be used as a photoinitiator. A three-level Box-Behnken design is used to explore responses with Design Expert (DE, Version 7.1, Stat-Ease Inc., Minneapolis, Minn., USA).

Each hydrogel formulation is assessed based on unconfined compression (ASTM D595), tensile test (ASTM D638). In addition, the chemical structures and thermal properties of the hydrogels are characterized with Fourier Transform Infrared Spectroscopy (FTIR), ¹H-NMR and Differential Scanning calorimetry (DSC). FTIR and ¹H-NMR are used to confirm the successful conjugation of BILs to gelatin structure. Gel Permeation Chromatography (GPC) is used to measure the molecular weight of hydrogels. The DSC is used to measure the glass transition temperature (Tg) of the hydrogels. Scanning Electron Microscopy is used to evaluate the physical structure of hydrogel with various formulation. Optimized hydrogel formulations are selected based on targeted degradation profiles (>7 days), low post-gelation swelling ratio (<50%), elasticity (˜50%) and mechanical stiffness (10-30 kPa). In the event of poor degradation profiles, copolymer additives, such as poly(ethylene glycol) diacrylate, can be incorporated into the prepolymer polymer formulation.

### Choline Functionalized PEG Diacrylate (PEGDA/BIL)

Choline functionalized PEG diacrylate (PEGDA/BIL) was synthesized by reacting choline acrylate and PEG diacrylate. The BIL was conjugated with the polymer at concentrations of 0%-20% (w/v) of polymer. The conjugation was carried out by mixing the BIL with a 25% (w/v) solution of PEGDA. The resulting polymer—BIL conjugate was then crosslinked by visible light induced photopolymerization, using LAP (lithium phenyl-2,4,6-trimethylbenzoylphosphinate) as photo-initiator to form the bio-adhesive that attaches to the surface and initiates the wound healing process. Lithium phenyl-2,4,6-trimethylbenzoylphosphinate (LAP) is a water-soluble, cytocompatible, type I photoinitiator typically used in the polymerization of hydrogels or other polymeric materials. This photo-initiator is preferred over Irgacure 2959 for biological applications due to its superior water solubility, higher polymerization rates with 365 nm light, and an absorbance at 400 nm allowing for polymerization with visible light.

The progress of the conjugation of the BIL to the polymers was tracked by NMR (FIG. 1I). The ¹H NMR spectra were collected to ascertain the conjugation of the BIL to the polymer. Methacrylate groups appeared in the conjugated polymer as characteristic peaks at ˜5.7 ppm and ˜6.1 ppm indicative of the conjugation of the polymer and ionic liquid leading to BIL incorporation in the polymer. This peak was absent in the spectrum for the non-BIL conjugated polymer. The appearance of a sharp peak at δ˜3.1-3.2 ppm in the conjugated polymer, corresponds to the three hydrogen atoms of choline (ammonium ion) and this also confirms the conjugation of BIL to the polymer.

### Example 2: In Vitro Cellular Cultures in Choline Functionalized Gelatin Methacrylate Hydrogel

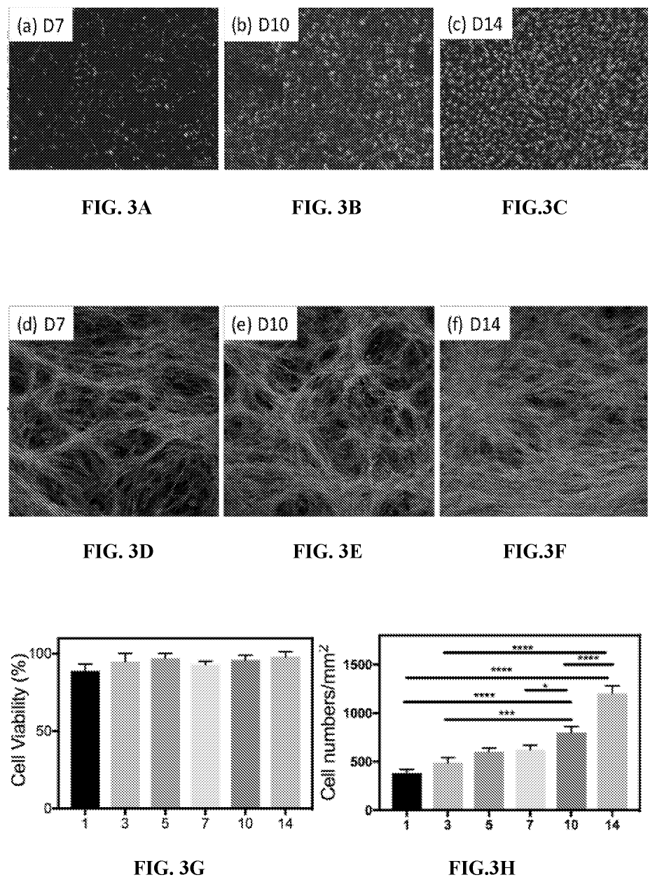

The potential of the choline functionalized gelatin methacrylate hydrogels to support the growth, spreading and function of primary C2C12 mouse myoblast cells was studied. A commercial live/dead assay (Invitrogen) was used to determine the viability of C2C12 cells growing on the surface of the hydrogel over a period of 14 days (FIGS. 3A-3C). Cell attachment and spreading in the hydrogel was evaluated through F-actin/DAPI immunofluorescent staining. The results demonstrated that the viability of C2C12 cells seeded on the hydrogels of the invention was not affected by the presence or absence of the choline functionalization (FIGS. 3G-3H).

Additional experiments are conducted with various GelMA/BIL hydrogel formulations to study interactions with 3T3 cells. 2D cell seeding is performed on the hydrogel surfaces at cell densities from 5×10⁶ to 1×10⁸ cells/ml. The cell viability is determined on days 1, 4, 7 and 14 by calcein-AM/ethidium homodimer Live/Dead assays. To quantify cell viability, the number of live and dead cells are counted by using the ImageJ software. Cellular attachment and spreading on surfaces of and within the 3D hybrid hydrogels are quantified.

### Example 3: Adhesion Properties of GelMA/BIL Hydrogels

The adhesion properties of various GelMA/BIL hydrogels were evaluated, modifying the concentration of the choline (0-20% w/v; FIG. 4). Introduction of 5% (w/v) BIL increased the adhesive strength by ˜40-45%, while incorporation of 20% (w/v) BIL increased the adhesive strength by nearly 350-360%. Without wishing to be limited to any particular theory, the enhancement in adhesion may be attributed to increases in the amount and concentration of electrostatic interactions. The improved film-forming properties is also attributable to the increase in the overall molecular weight due to the addition of bulky choline side chains. While GelMA by itself exhibits some amount of polar and hydrogen bonding interactions, the introduction of choline based functionalities significantly increases the amount of strong electrostatic interactions leading to high adhesive strength. The increased amount of potential electrostatic interactions is advantageous for application as an adhesive hemostatic bandage, wherein the material would be expected to adhere to the site of a would due to interaction between the cationic choline “head” and the phospholipidic bilayers of exposed cells.

Similar testing is conducted on additional GelMA/BIL hydrogel formulations having varied ingredient concentrations and crosslinking conditions, as outlined in Example 1. Standard wound closure, lap shear, cyclic test and burst pressure tests, as reported in Assmann, et al. Biomaterials Volume 140, September 2017, Pages 115-127, are used to study adhesion properties of the GelMA/BIL hydrogel formulations. Optimized hydrogel formulations are selected based on adhesion strength (>100 kPa), shear strength (>500 kPa), burst pressure (>5 kPA) and cyclic (>12000 cycles). Adhesion tests are conducted using non-functionalized GelMA, COSEAL® and EVICEL® sealants as controls.

### Example 4: Ex Vivo Testing of GelMA/BIL Hydrogel Adhesive Hemostatic Bandage

A fresh porcine heart is obtained to perform the long-term cyclic analysis. A circular defect of 4 mm diameter is created with biopsy punch on the atrium wall of the heart. The defect is then sealed with an 8 mm-diameter disc of a GelMA/BIL hydrogel adhesive. The heart with the attached sealant is submerged in a large reservoir of PBS. The inflation-deflation deformation of the heart is mimicked by pumping air in and out for 2-18 hours (2000-20,000 cycles in total). The burst pressure tests are done by visceral pleura defect. Before testing the sealants, the tissues are evaluated for air-leak after the creation of 1 cm circular defects in the visceral pleura. Air-leak is confirmed by submerging the lungs in PBS. Pleural defects are sealed using the ten selected GelMA/BIL sealants, with GelMA hydrogel, COSEAL® and EVICEL® as controls. Lobes are then ventilated for 2 min at an airway pressure of 10 cm H₂O. Airway pressure is be increased by five cm H₂O at 2 min intervals. Lungs are submerged in a bath containing PBS at 37° C. to test for air-leak at each interval. This process is continued until air leak is observed and the burst pressure is the minimal peak pressure at which gas bubbles are observed. The burst pressure value of the selected sealant candidates is also compared with controls. The interface between the engineered sealants and the lung tissue is compared using SEM images from the freeze-dried samples or by performing H&E staining on the sealant/tissue samples.

### Example 5: In Vitro Antimicrobial Properties of GelMA/BIL Hydrogel

Bacterial infection can potentially lead to acute infection and sepsis or can result in osteomyelitis, which is life-threatening. Thus, controlling infection is a critical step in managing trauma patients. Inhibition of bacterial growth at an injury site can eliminate the need for systemic antibiotic administration. As the bacteria related to trauma associated injuries are diverse, an antimicrobial agent should cover a broad spectrum of bacteria including resistant organisms and have a low minimum inhibitory concentration (MIC) (with high cytocompatibility) and ideally the agent should not lead to the development of resistant bacteria. The bacteria involved in trauma associated injuries are predominantly gram-negative anaerobic bacteria and may include A. actinomycetemcomitans, P. gingivalis, P. intermedia, B. forsythus, C. rectus, E. nodatum, P. micros, S. intermedius, Acinetobacter baumannii and Treponema sp.

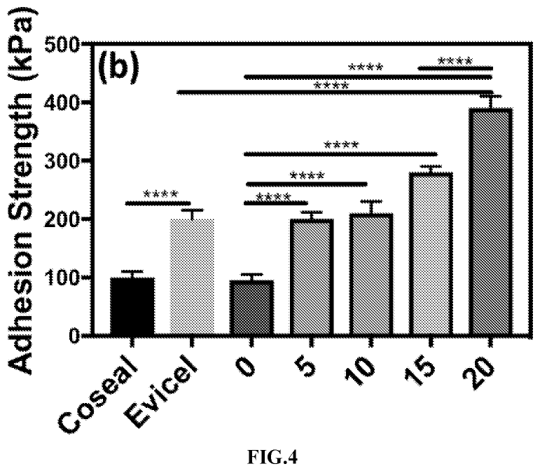

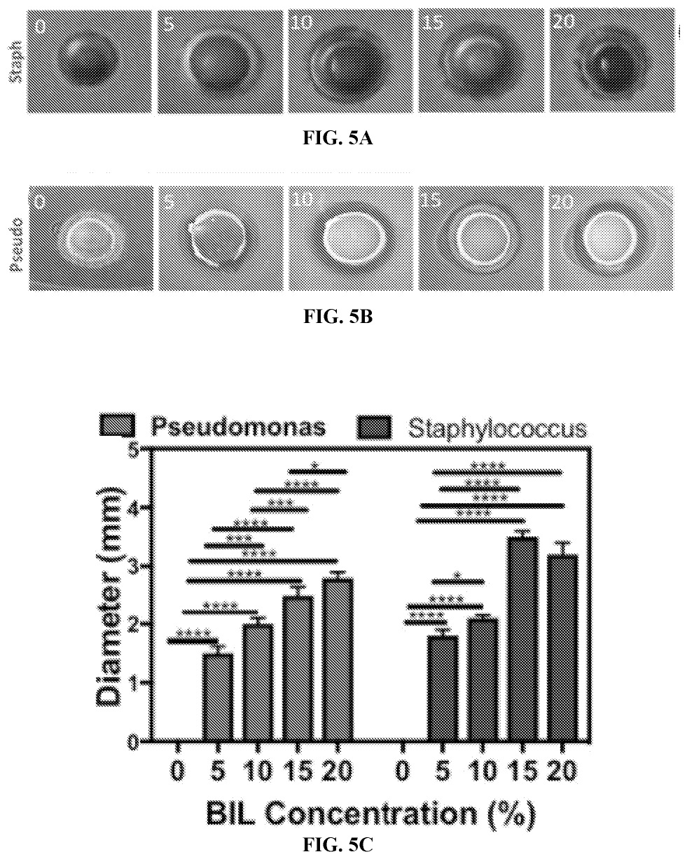

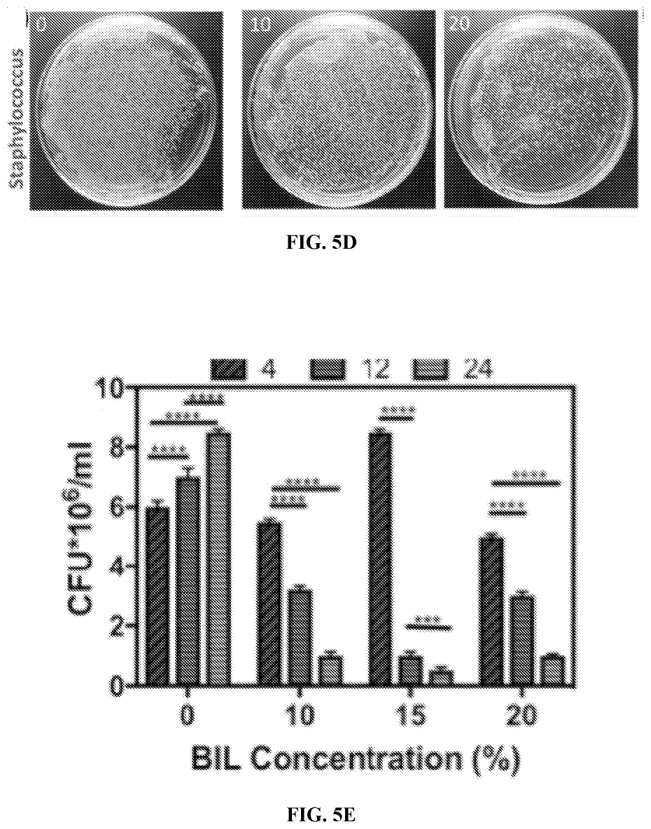

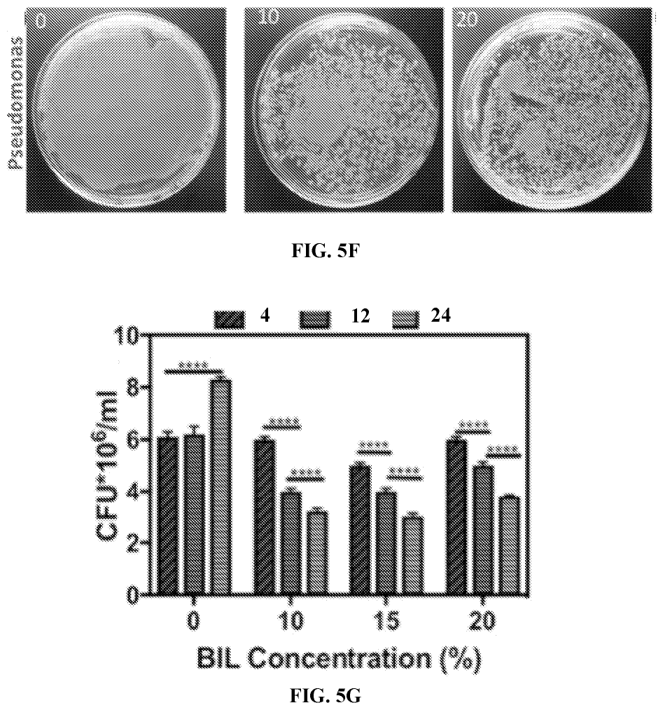

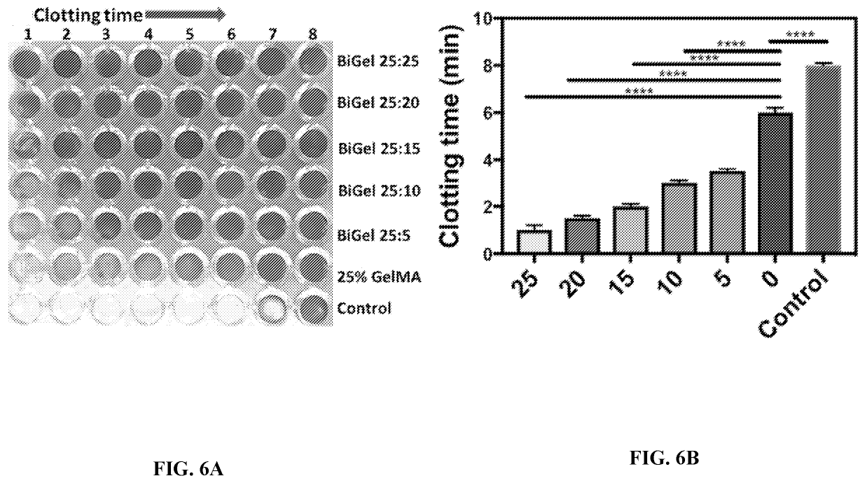

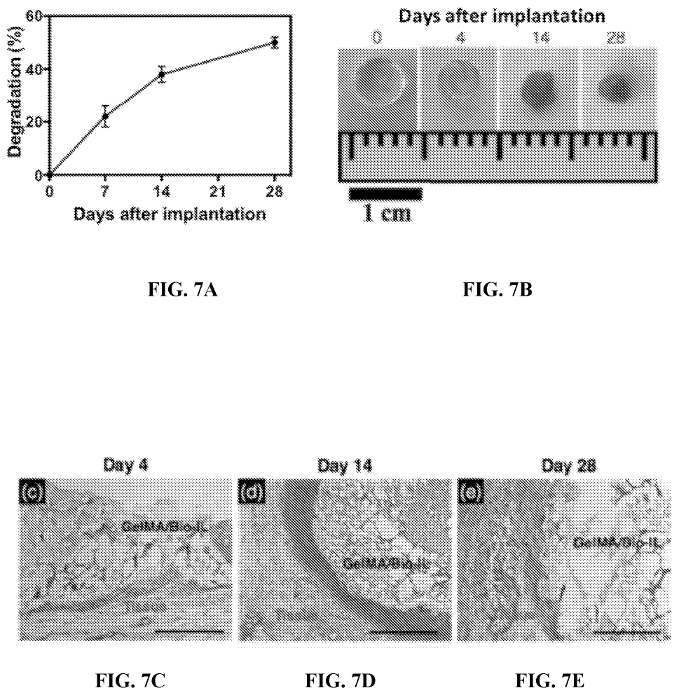

The antimicrobial activity of the GelMA/BIL hydrogels was studied in in vitro antibacterial assays using two bacterial strains: Pseudomonas and Staphylococcus. GelMA/BIL hydrogels with BIL concentrations ranging from 0-20% (w/v) were placed in agar plates with each bacterial strain. FIGS. 5A-5B are images showing inhibition of bacterial growth for the Staphylococcus and Pseudomonas strains respectively. FIG. 5C is a graph showing the diameter of the disc surround the polymer droplet, representing bacterial growth inhibition plotted against the extent of choline functionalization of the hydrogel. The results showed that the capacity for bacterial growth inhibition increased as a function of increasing choline functionalization. However, a tapering effect was observed wherein choline concentration above 15% (w/v) provided minimal benefit. The unfunctionalized control did not demonstrate any appreciable bacterial growth inhibition for either strain.

The results of a colony forming unit assay are shown in FIGS. 5D and 5F and are enumerated in FIGS. 5E and 5G. The photographs of the extent of colony formation after an incubation period of 24 hours is shown in FIGS. 5D and 5F for Staphylococcus and Pseudomonas respectively. With both bacterial strains, greater inhibition of colony formation was demonstrated for GelMA having greater choline functionalization. Optical density (OD) measurements were taken at 4, 12 and 24 hours confirming this finding. Optical density rose rapidly for control plates having unfunctionalized GelMA. GelMA/BIL hydrogel having 10% (w/v) BIL demonstrated nearly a 50% reduction in optical density between 4 hours and 12 hours and 85% between 12 hours and 24 hours for the Staphylococcus assay. GelMA/BIL hydrogel having 15% (w/v) BIL demonstrated nearly 95% reduction in optical density between 4 hours and 24 hours for the Staphylococcus assay. As was observed in the agar disc assays, a tapering effect was observed between 15% (w/v) and 20% (w/v) BIL where a smaller marginal benefit was observed at short time points and a net decrease in effectivity was observed over longer time periods for GelMA/BIL hydrogels having higher choline functionalization. With the Pseudomonas strain, the % reduction in OD obtained over a 20 hr period (4 h to 24 h) with an increase in BILs (% w/v) from 5 to 20 (% w/v) are monotonic and less stark than those obtained with Staphylococcus.

Additional assays are conducted utilizing similar protocols to test the antibacterial properties of GelMA/BIL hydrogels. Bacteria strains A. actinomycetemcomitans, P. gingivalis, P. intermedia, B. forsythus, C. rectus, E. nodatum, P. micros, S. intermedius, Acinetobacter baumannii and Treponema sp. are cultured on Mueller-Hinton agar plates until the bacterial growth fully covers the entire area of the plate. Then GelMA/BIL hydrogel as described in Example 1 are placed on top of the bacterial-grown agar plate. Based on the antibacterial activity of the hydrogels demonstrated in the preliminary results, the bacterial growth surrounding the hydrogels can be cleared, and in certain embodiments the clearance area is proportional to the amount of applied hydrogel and choline functionalization.

Colony forming unit (CFU) assays are also used to evaluate antimicrobial activity of the GelMA/BIL hydrogel against the additional strains discussed above. The bacteria are cultured overnight in an 37° C. incubator in Mueller-Hinton agar (MHA; Difco) until the colonies appear on MHA. Subsequently, two colonies are harvested and grown overnight in Mueller Hinton Broth (MHB; Difco) while shaking in the incubator at 37° C. To obtain bacteria in the mid-logarithmic phase of growth, 100 μL of the original bacterial solution are transferred into five mL of Mueller Hinton Broth (MEM) and incubated at 37° C. for 1 h. The bacteria suspension is diluted again with MEM to reach to a final concentration of ˜106 cells/mL, as determined using a spectrophotometer. Briefly, the bacteria culture is measured to reach OD₆₀₀ 0.3-0.5 after calibrating the spectrometer with MEM to prepare a mid-Log (exponential phase) suspension culture. The GelMA/BIL hydrogel candidates from Example 1 are placed in a 12-well culture dish, rinsed with PBS, and two mL of bacterial solution (density ˜106 bacteria/mL) are dropped into each well. Unfunctionalized GelMA hydrogel is used as the negative control. The samples are incubated for 1, 4 and 8 h at 37° C. humidified incubator with 5% CO₂, 20% O₂, while shaking at 70 rpm. The residual bacteria in 10 μL are plated on nutrient MHA agar and incubated overnight at 37° C. The surviving bacteria for each specimen are evaluated by measuring colony-forming units (CFU) by counting the number of bacteria colonies on a plate with a countable number of colonies after serially diluting the bacteria solution.

### Example 6: In Vitro Hemostatic Properties of GelMA/BIL Hydrogel

As shown in FIGS. 6A-6B, clotting time assays were conducted to determine the effect of GelMA/BIL hydrogels on the clotting of whole blood. The results showed that the extent of clotting and the speed of clot formation increased as a function of increasing choline functionalization. The negative control, having unfunctionalized GelMA exhibited a 22-24$ reduction in clotting time. Clotting rate increased as a function of increasing choline functionalization. A choline concentration of 5% (w/v) demonstrated a reduction of clotting time of 63% over a control having no GelMA and nearly 48-50% reduction of clotting time over the unfunctionalized GelMA control. As the relative amount of choline functionalization increased, the rate of clotting increased but each increase in choline concentration yielded a smaller marginal increase in clotting rate. The overall reduction in clotting time with 25% (w/v) BIL was nearly 83% over the unfunctionalized GelMA control.

Further clotting time assays are conducted on additional GelMA/BIL hydrogel formulations as outlined in Example 1. Briefly, citrated blood (630 μl) is added to a 1.5 mL Eppendorf tube. A total of 70 μl of 0.1 M calcium chloride are added and the solution is vortexed for 10 s. 50 μl of the citrated blood solution are added to sequential wells of a 96 well plate. Each well is coated in a GelMA/BIL hydrogel formulation or a control composition (e.g. unfunctionalized GelMA, no polymer, thrombin containing wells). At selected time points, each well is washed with 9 g/L saline solution to halt clotting. The liquid is aspirated after clotting is stopped and washes are repeated until the solution becomes clear, indicating removal of all soluble blood components. Final clotting time is determined as the well that forms a uniform clot wherein all subsequent wells at later time points with the same hydrogel formulation have no change in clot size.

### Example 7: Absorption of Red Blood Cells (RBCs) by GelMA/BIL Hydrogel

GelMA/BIL hydrogel formulations as outlined in Example 1 are added to a known weight of RBC pellet and the resulting suspension is mixed by inversion at room temperature for 1 h. After centrifugation (2,000 rpm for ˜5 min), the activity of the supernatant is measured by ESI-TOF-MS spectroscopy. The difference between total activity of the solution before and after adding GelMA/BIL hydrogel is used to determine the amount of hydrogel bound to the RBC surfaces. Activity is corrected for dilution by trapped buffer volume. The amount of hydrogel bound per cell can be calculated from the activity of bound hydrogel, the specific activity of the hydrogel, the number average molecular weight of the hydrogel and the density and volume of the RBCs.

### Example 8: In Vivo Biocompatibility and Degradation of GelMA/BIL Hydrogel Sealants in a Rat Subcutaneous Model

In order to test in vivo biocompatibility and degradation, GelMA/BIL hydrogel sealants are tested alongside unfunctionalized GelMA hydrogel as an implant in a rat model. Each implant is observed at four different time periods: 3, 7, 14 and 28 days. The biocompatibility of samples is determined based on the presence of inflammatory markers including lymphocytes (CD3) and macrophages (CD68).

Anesthesia is achieved by 2.0 to 2.5% isoflurane inhalation, followed by 0.02 to 0.05 mg/kg SC buprenorphine administration. After inducing anesthesia, eight 1-cm incisions are made on the posterior medio-dorsal skin, and small lateral subcutaneous pockets are prepared by blunt dissection around the incisions. Hydrogels (1×5 mm disks) were implanted into the pockets, followed by anatomical wound closure and recovery from anesthesia. Animals were euthanized by anesthesia/exsanguination at days 3, 7, 14 and 28 post-implantation, after which the samples were retrieved with the associated tissue and placed in DPBS.

In order to determine biocompatibility of the hydrogels, histological analyses are performed on cryosections of the explanted hydrogel samples in order to characterize the inflammatory response elicited by the implanted material. After explantation, samples are fixed in 4% paraformaldehyde for 4 hours, followed by overnight incubation in 30% sucrose at 4° C. Samples are then be embedded in Optimal Cutting Temperature compound (OCT) and flash frozen in liquid nitrogen. Frozen samples are then sectioned using a Leica Biosystems CM3050 S Research Cryostat. 15-μm cryosections are obtained and mounted in positively charged slides using DPX mountant medium (Sigma). The slides are then processed for hematoxylin and eosin staining (Sigma) according to instructions from the manufacturer. Immunohistofluorescent staining is performed on mounted cryosections. Anti-CD3 [SP7] (ab16669) and anti-CD68 (ab125212) (Abcam) are used as primary antibodies, and an Alexa Fluor 594-conjugated secondary antibody (Invitrogen) is used for detection. All sections are counterstained with DAPI (Invitrogen), and visualized on an AxioObserver Z1 inverted microscope (Zeiss).

### Example 9: In Vivo Evaluation of GelMA/BIL Hydrogel Sealants in Traumatic Surgery

To determine the functionality of the engineered hydrogel materials as robust sealants for clinical applications, in vivo tests are performed using small and large animal models. The sealants are evaluated for their ability to seal, repair and provide hemostasis to traumatic injury defects.

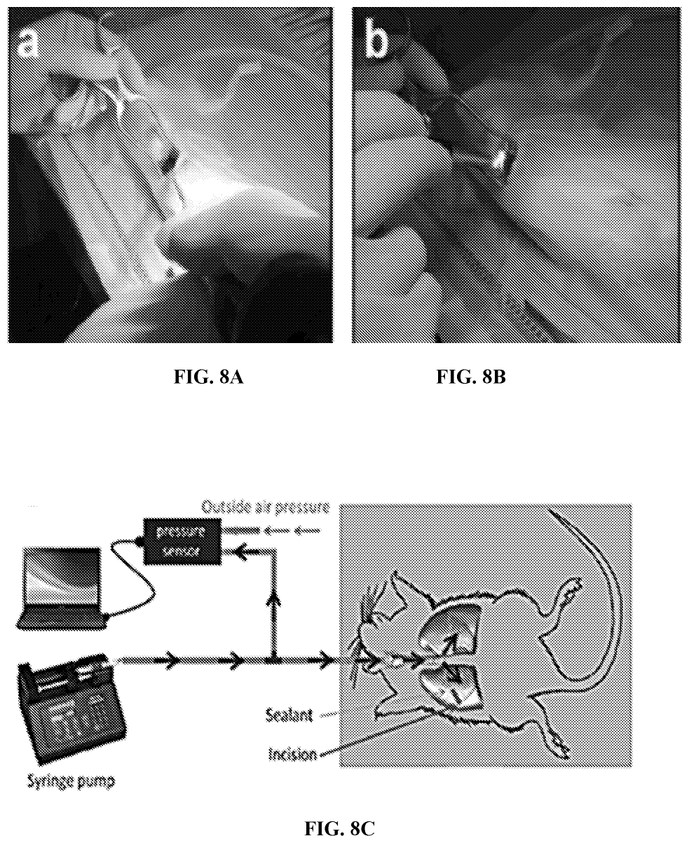

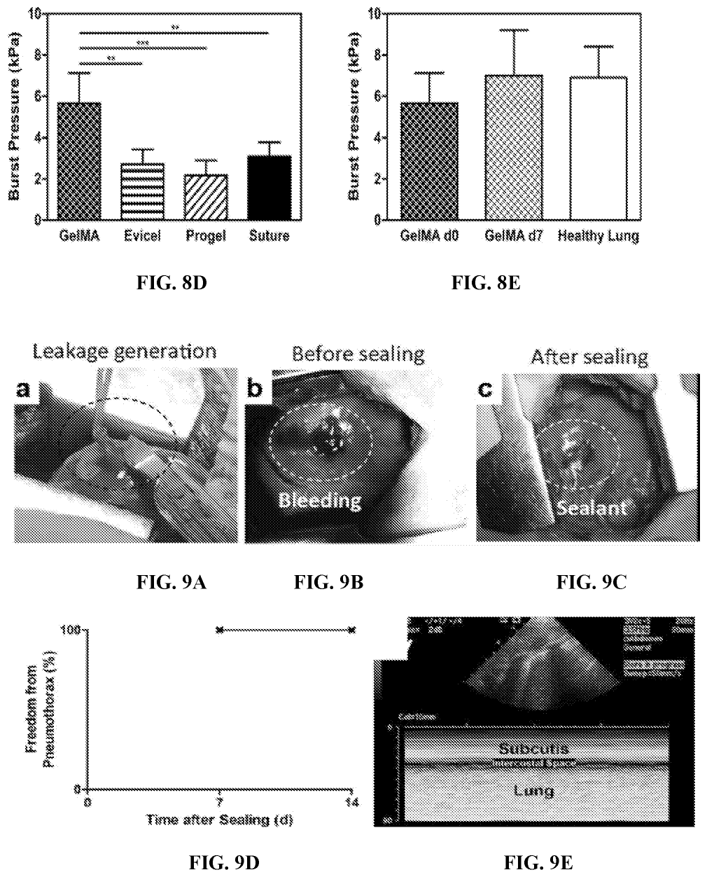

In a preliminary study, in vivo experiments were performed using UV light crosslinkable unfunctionalized GelMA hydrogel in two animal models, a lung incision rat model (FIGS. 8A-8E) and a lung incision pig model (FIGS. 9A-9E). In both models, incisions were created on the tissues and unfunctionalized GelMA was applied and cured in situ to seal the incisions. These models are used as the basis for testing the GelMA/BIL hydrogel formulations as outlined in Example 1.

A rat model of standardized lung leakage was established to test the suitability and effectiveness of GelMA sealant for pulmonary lesions in the absence of any additional convention surgical methods such as suturing or stapling (FIGS. 8A-8E). The initial sealing strength and postoperative performance (up to 28 days) of GelMA during autologous defect repair was examined. All rats survived the surgery and the follow-up period. No clinical signs of postoperative pneumothorax were observed, and at the end of the follow-up period, no lung leakage was found.

In order to test the function of the engineered GelMA sealants in a larger animal model, a swine visceral pleura defect model was developed, which closely resembles the human visceral pleura (FIGS. 9A-9E). All pigs survived the surgery and the follow-up period of 14 days. No clinical or sonographic signs of pneumothorax were observed during the follow-ups (FIGS. 9C-9D). After 14 days, the defect sites were covered by connective repair tissue, while GelMA had disappeared (FIG. 9E).

To further evaluate the adhesion and hemostatic properties of GelMA/BIL hydrogels, rat liver bleeding and tail amputation models are used for assessment of the hydrogel function as a topical hemostatic agent. Surgeries are completed on 1˜2-month-old Sprague-Dawley rats (Male and Female), and anesthesia is induced with 10% chloral hydrate by intraperitoneal injection (0.5 mL/100 g). Tail amputation is completed at 5 cm from the tail base using surgical scissors. The hydrogel is applied, and the volume of blood loss is measured by the use of the optimal case and is compared with the values for the non-treated group. The liver injury model is conducted by placing a cut on the rat liver about 2 mm in depth with a surgical blade. Adhesive hydrogel (˜2 g) is placed on the liver to seal the cut, and blood loss and animal survival are compared between the treated group and the non-treated group. Rats are placed in 4 groups: 1) GelMA/BIL hydrogel, 2) unfunctionalized GelMA, 3) no adhesive, and 4) Thrombin treated.

Additionally, a penetrating inferior vena cava injury in coagulopathic swine model is used to assess the efficacy of a topic hemostatic agent to control hemorrhage. Briefly, Yorkshire pigs (body weight 40-50 kg, male and female) are used to evaluate the performance of the GelMA/BIL hydrogel formulations. Pigs are placed in 4 groups: 1) GelMA/BIL hydrogel, 2) unfunctionalized GelMA, 3) no adhesive/suture repair, and 4) Thrombin treated. The pigs receive a 25% exchange transfusion of blood for refrigerated normal saline to induce a hypothermic coagulopathy. A laparotomy is performed and a standardized 1.5 cm injury to the inferior vena cava is created. Animals are randomized into one of 4 groups, as outlined above; 3 adhesive groups and 1 control group which has a suture repair of the injury. Following adequate hemostasis, the laparotomy incisions are closed and the animals are recovered. Animals are maintained for 28 days in a vivarium and given access to food and water ad lib, as well as pain medication. Animals are then euthanized, and the tissue of the area of injury is assessed for healing. All procedures are performed under general anesthesia with subjects intubated and ventilated (10 cc/kg IBW, 20 bpm) during the whole procedure. Induction anesthesia is achieved with ketamine (20 mg/kg IM dose) and versed (0.5 mg/kg IM dose) followed by isoflurane 5% induction via face cone. Once appropriate sedation has been achieved, the animals are intubated and provided a maintenance of isoflurane at 1-4%. Vital signs are assessed throughout the procedure to determine if the animal is experiencing any pain and anesthesia is titrated to ensure animal comfort. Postoperative pain control is achieved with buprenorphine (0.01-0.02 mg/kg IM route) up to twice daily as needed.

Interval follow-up after surgery is used to detect the failure of the materials as a surgical sealant for reducing pleural defects. The number of blood-leaks is evaluated with thoracic ultrasound and recorded on day 3 and day 14. Pigs are euthanized via Fatal-Plus on 28, and the tissue with the applied sealant is isolated. The tissue around the defect is evaluated for any defects. The explanted tissues are tested for burst pressure. H&E staining for gelatin, collagen, and fibroblast is also performed on cross-sections of the center and edges of the pleural defects.

### Example 10: Characterization and Comparison of In Vitro Swelling, Degradation, and Mechanical Properties of of BioGel and BioPEG

In vitro swelling, mechanical properties, and the in vitro degradation rate of the polymer/BIL was evaluated. Hydrogels can be metabolized through various pathways. Hydrogels can decompose in the presence of enzymes, which recognize and attack specific functionalities on the hydrogel backbone. Hydrogels can also decompose by cells or simply via hydrolysis at either acidic or basic pH conditions. In order for the hydrogel to mimic in vivo conditions, to prevent its rejection as a foreign invasive object, prevent infection, and be eventually metabolized it should be adequately hydrated via water uptake from body fluids. In the context of a biopolymer based adhesive or in tissue engineering, excessive water uptake and degradation could potentially lead to impaired mechanical and adhesive properties. Thus the water uptake and swelling for a tissue adhesive must be optimized to suit the in vivo conditions and the intended application.

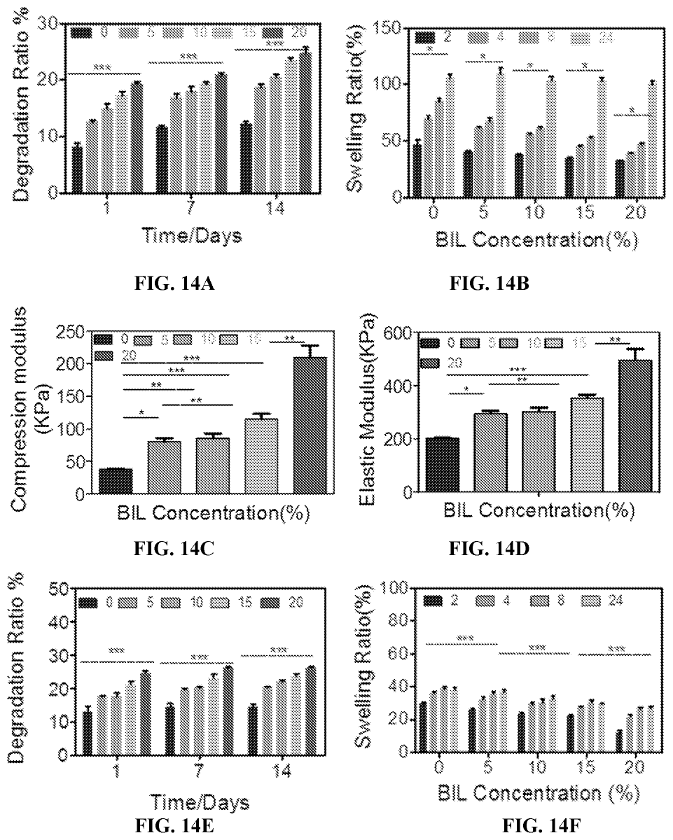

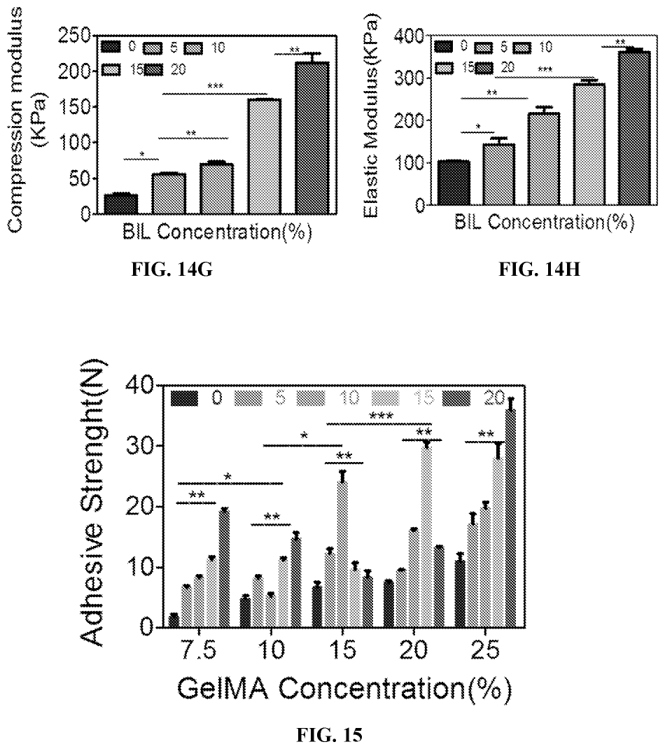

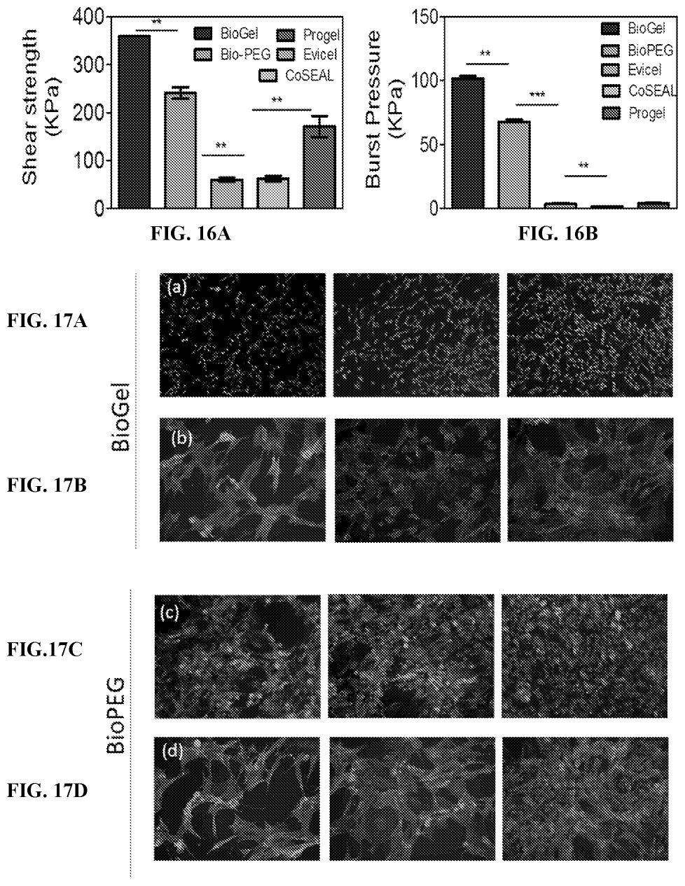

The swelling of BioGel versus the BIL loading in the polymer is shown in FIG. 14B. Irrespective of the BIL loading in the polymer, the BioGels grew to approximately the same levels of 120% swelling ratio. For the GelMA (0% BIL loading), there appeared to be a steady increase in the water uptake and swelling over a period of time, whereas for the polymers containing BIL loadings of 5, 10, 15 and 20%, there appears to be a gradual uptake of water in the first few hours, followed by a sudden increase to reach around 120% at the end of 24 hours. This effect is progressively more pronounced as the BIL concentration increases. In this context, the fundamental response of a polymer in its absorption pattern of water may be called into understanding. As the polymer absorbs water, in a semi-dilute solution, polymer chain conformations form overlapping correlation blobs with hydration spheres around each blob dictated by functional groups with water affinity. For GelMA, this is relatively straightforward, with no functionalization, the semi-dilute regime blobs slowly open up in a favorable solvent and allow more and more water in till the crosslink conformation resists any further expansion. With the advent of the BIL functionalization, there is an increased initial strong interaction between monomers inside a single correlation blob, as well as between the monomers in the periphery of the correlation blob which act as additional “cross links” conferring an initial resistance to polymer expansion upon increased hydration. This needs to be slowly broken by hydrating the hydrophilic groups intrinsically in the GelMA as well as in the BioGeL conferred by the BIL functionalization. Hence, the rate at which the water expands the gel is slower in BioGel rather than in GelMA. However, the GelMA is already highly hydrophilic the addition and variation of BIL loading does little to alter that. It should be noted that these are crosslinked polymers, crosslinked to the same extent using LAP, hence the final expansion of such a polymer would eventually be arrested by the existence of those chemical crosslinks acting as the final tethers. Thus for polymers, the density of crosslinking determines the final swelling and hence the polymers swell to a limited extent and to the equal extent here. This is a desirable property when designing an adhesive formulation wherein, the material must swell and mimic in vivo conditions, but not to the extent that it starts to disintegrate and defeat the primary mechanical requirement of usage.

A similar trend was seen with BIL incorporated PEGDA (BioPEG) (FIG. 14F). With 0% BIL incorporation, the polymers swelled much faster than when BIL was incorporated. With increasing BIL incorporation, the swelling rate decreased due to initial intrinsic physical crosslinking induced by electrostatic interactions mediated by the BIL functional groups. The final swelling achieved for the PEGDA backbone based BioPEG polymers ˜40% is lower than that for GelMA based polymers due to the intrinsic nature of the PEGDA backbone which has lower hydrophilic affinity than GelMA.

In vitro degradation tests can simulate the in vivo behavior of hydrogels when exposed to physiological conditions. Hydrogel degradation studies are also important to ensure that the material degrades into safe components, which can be identified and further analyzed to assess their toxicity in cell hosts. Due to its organic nature, the degradation of BIL functionalized polymer is not expected to generate any cytotoxic byproducts. The polymers, with and without BIL conjugation were incubated in Dulbecco's phosphate-buffered saline (DPBS) at 37° C. for 14 days. Degradation studies (FIG. 14A) show that for BioGel with BIL loading of 0%, the degradation increases from 8.5% at the end of day 1 to 12.3% at the end of day 14, the degradation tapers after 7 days. There is a general increase in the amount of degradation at the end of day 1, day 7 and day 14 with an increase in the amount of BIL loading. For 5% BIL loading, the degradation moves from 13% at the end of day 1, through around 18% at the end of day 7, tapering off at 19% at the end of day 14. The degradation for 20% BIL loaded polymer is 19.% at the end of day 1 and reaches 24.6% on day 14. While the trend of degradation is the same, it may be observed that there are greater levels of degradation with an increase in BIL loading. It is possible for the quarternary ammonium head in choline to act as a catalyst along with the water of hydration to cause hydrolysis of the many integrated functionalities in both the GelMA and PEGDA backbones, hence degrading those. This is the reason why degradation shows an increasing trend with BIL loading in the polymer. Degradation follows the same trend in both GELMA (FIG. 14A) and PEGDA (FIG. 14E) based polymers but the lower water uptake in PEGDA results in an intrinsically lower extent of degradation.

Mechanical properties of composite hydrogels were characterized through tensile and cyclic compression tests. The elastic moduli obtained from tensile tests are shown in FIGS. 14D and 14H. Tensile tests on BioGEL (GelMA+BIL) and BioPEG (PEGDA+BIL) hydrogels revealed that the elastic moduli (FIGS. 14D and 14H) and compression moduli (FIGS. 14C and 14G) of the engineered hydrogels could be modulated by varying the % of BIL. As a general trend, the mechanical properties increase with increase in BIL's concentration. With 0% BIL in the BioGEL, the compression modulus is 37.797±0.471 KPa and a tensile modulus of 202.873±1.264 KPa. These values increase to 186.466±7.506 KPa and 355.365±18.252 KPa respectively for BioGEL with 20% BIL loading. Similarly, BioPEG with 0% BIL loading (PEGDA) shows a compression modulus of 26.12±2.634 KPa and a tensile modulus of 102.932±1.611 KPa, which increase to 212.157±13.113 KPa and 361.213±7.246 KPa respectively at 20% BIL loading in BioPEG. Hydrogels are expected to exhibit an optimum tradeoff between stiffness and flexibility compared to the tissues to resist shear, tension or compression forces while maintaining structural integrity. These results demonstrated that the bio-adhesive synthesized could be tuned to possess high elasticity and durability. The increase in compressive strength and tensile modulus of the BioGEL and BioPEGs with BIL loading over unfunctionalized GelMA or PEGDA can be attributed to an increase in the intensity of electrostatic interactions. A further reason for this increase is the attachment of the bulky choline based functionalization as side chains to the GelMA or PEGDA structure which makes the backbone less able to rotate due to increased stiffness. This reduces the labile nature of the polymer hence increasing the respective moduli. Enhanced mechanical properties is also a result of an increase in the overall molecular weight due to bulky choline side chains. GelMA and PEGDA with 0% BIL has some polar and hydrogen bond based interactions which increase significantly, in addition to the increased electrostatic interactions by the addition of BIL functionalization. An increase in these strong electrostatic interactions obstructs the uncoiling and slipping of chains. This results in the BIL functionalization acting as a physical crosslink, tethering the structure together and hence the enhanced moduli.

### Example 11: In Vitro Adhesive Properties of BioGel and BioPEG

Typical methods of wound closure are associated with disadvantages including stress and tissue damage. In order to mitigate these complications, BioGel and BioPEG based adhesive materials can be an alternative strategy. The in vitro lap shear, adhesive and burst strength tests, and the response to shear, compression, or extension as well as high pressures upon the adherence of the gel to tissue were characterized, all in accordance to ASTM F2255-05 standard.

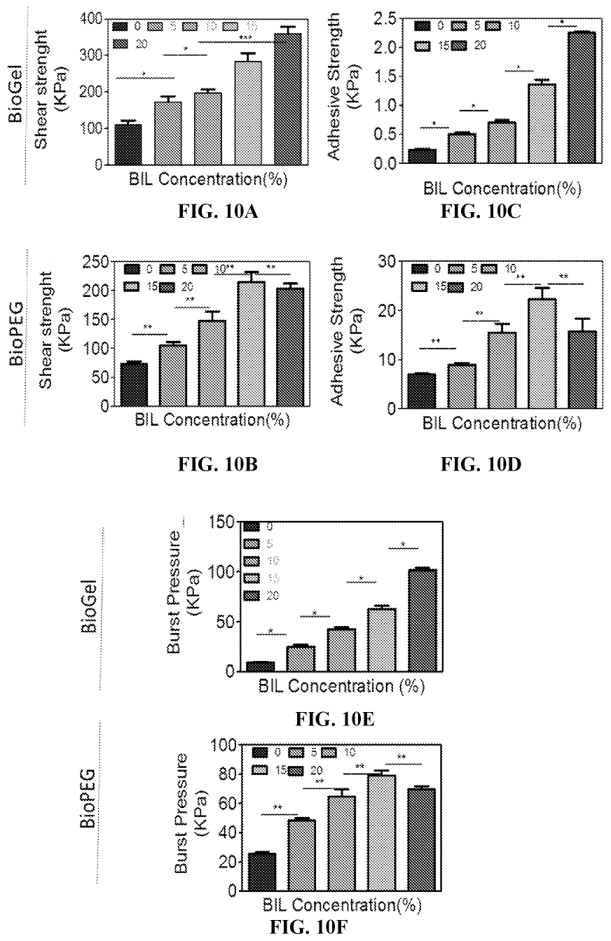

FIGS. 10A-10B show the shear strength of BioGEL and BioPEG with increasing BIL loading. The shear strength of BioGel increased from 109.633±12.42 KPa (GelMA with 0% BIL) to 359.393±18.72 KPa for BioGEL with 20% BIL loading. Similarly, for BioPEG the shear strength increased from 73.4±3.84 KPa for the polymer with 0% BIL loading to 241.00±12.097 KPa at 20% BIL loading. The adhesive strength is shown in FIGS. 10C-10D. The adhesive strength of GelMA with 0% BIL loading was measured to be 0.233±0.0166 KPa and it increased to 2.253±0.0240 KPa for BioGEL 20% (w/v) BIL. Similarly, the adhesive strength of PEGDA, with 0% BIL loading was measured to be 7.0±0.2 KPa and it increased to 38.7±0.3 KPa for BioPEG with 20% BIL loading.

The shear strength and adhesive strength of BioGel and BioPEG with high BIL loadings are significantly higher compared to reported values for commercially available tissue adhesives such as Ethicon's Evicel and Baxter's Coseal. The shear strength of Evicel and Coseal are 207.65±67.3 kPa and 69.7±20.6 kPa respectively while the adhesive strength for these are 1.94±0.99 kPa and 1.68±0.11 kPa) respectively.

The burst pressure test establishes the ability of an adhesive to withstand the pressure exerted by underlying tissues and fluids from within the wound site. Burst pressure on the engineered composite hydrogels was tested, based on a variation of the ASTM F2392-04 standard testing for surgical sealants. The results are shown in FIGS. 10E-10F. Burst pressure for BioGel and BioPEG at 0% BIL loading was 9.173±0.663 KPa and 25.603±0.998 KPa respectively. This subsequently increased to 101.742±2.12 KPa and 69.415±1.585 KPa, respectively, for BioGEL and BioPEG at final BIL loading of 20%. These values were also significantly higher than that of currently available tissue adhesive.

Adhesive hydrogel design entails tailoring properties to ensure high tissue adhesion and appropriate mechanical strength. Hydrogel adhesives for soft tissues need mechanical characteristics comparable to native tissue to ensure proper tissue movement. The adhesion properties should be high enough to enable attachment to the surrounding tissues. The adhesive material should be biodegradable with a degradation rate relative to tissue ingrowth and exhibit high biocompatibility. It was inferred, from the results that the introduction of BIL functionalization to GelMA or PEGDA improves the adhesion of the hydrogel to tissues especially in under in vivo conditions.

The adhesive property is directly related to electrostatic interactions. It is also related to better film-forming properties which increase with increasing overall molecular weight. The molecular weight, in turn, is dependent on the average molecular mass of repeat units in the polymer which increases with increasing functionalization by bulky choline pendant groups. Both GelMA and PEGDA on their own are good film formers and they already have the allowance for polar and hydrogen bond based interactions. However, the introduction of choline BIL based side groups significantly increases these strong electrostatic interactions leading to high adhesive and shear as well as burst pressure strength. It is also expected that the surface BIL heads of the adhesive layer will interact with the phospholipidic bilayers of the exposed cells of the cornea, wherein the polar heads may be expected to enhance adhesion.

### Example 12: Characterization of In Vitro Hemostatic Properties of BioGel and BioPEG

Hemostatic properties of adhesive material are expected to enhance the efficacy of adherence to wet surfaces and reduce superficial corneal and general tissue blood loss without interfering with vasculature development. Despite there being several current techniques to impart hemostasis, there are few that can be considered ideal, affordable and easily usable in topical applications without immunogenic and pathologic thrombotic side-effects. Certain studies have studied hydrogels for blood clotting, in particular, cationic hydrogel which forms a physical barrier to blood loss by forming aggregates and shear thinning nanocomposite hydrogels with silicate nanoplatelets and gelatin as injectable hemostatic agents. However, there exists a tradeoff between hemostatic properties and adhesive properties and the current materials exhibit very poor adhesive properties.

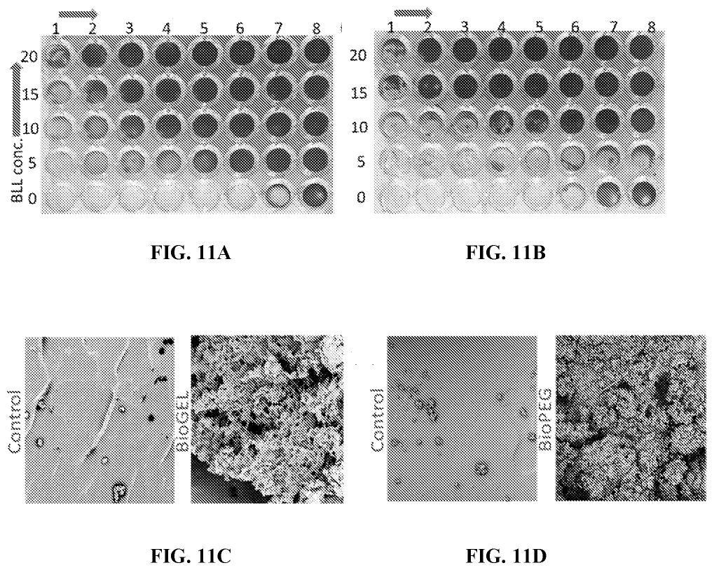

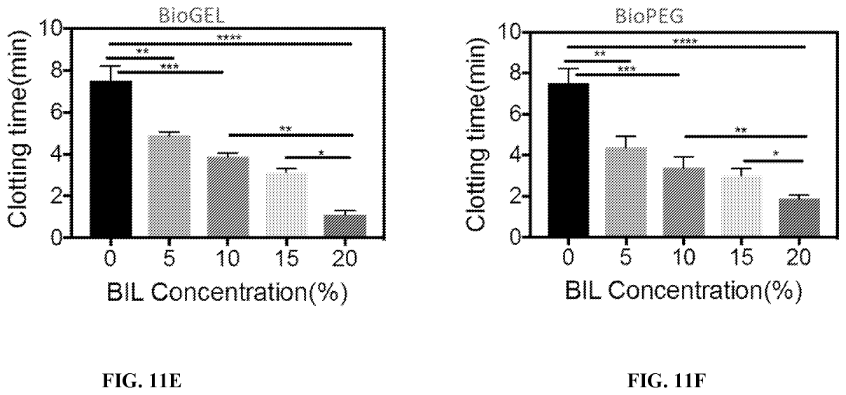

The coagulation properties of BioGEL and BioPEG with BIL loading varying from 0%-20% BIL loading were studied and the results are shown in FIGS. 11A-11F. There is a significant increase in the extent and rapidity of clot formation with increasing BIL functionalization. With GelMA and PEGDA, with 0% BIL functionalization, a slight reduction in clotting time was seen compared to the control as shown in FIGS. 11A-11B. With a further increase to 5% BIL functionalization, the clotting time decreases from 7.5±0.500 mins to 4.875±0.125 mins. Further increase in the concentration of the BIL to 20% (w/v) decreases the clotting time to 1.125±0.125 mins indicating an increase in coagulation efficiency with increasing BIL concentration. Similar results were demonstrated in the case of BioPEG. When the bioadhesive samples were incubated with RBC pellet overnight to perform SEM on the samples, (FIGS. 11C-11D) the images indicated that with the increasing concentration of the BIL the coagulation of the RBC's increased.

Cell membranes are known to consist of a phospholipid bilayer of which 1,2-dipalmitoyl-glycero-3-phosphatidyl choline (DPPC) is a major constituent. Choline functionalization imparts a quarternary ammonium moiety—the cholinium head group—which, over the mechanism of cellular adhesion, interacts with phosphatidyl choline groups, forming quaternary nitrogen-phosphorus pairs, creating a quadrupole with high electrostatic forces.

When macromolecules and cellular surfaces interact, the former adsorb and bring the surfaces into greater proximity in a bridge conformation. Also, the exclusion of glycocalyx from the intercellular space pushes the cells together owing to the osmotic pressure gradient. The same mechanism, when applied to red blood cells cause their distortion due to overcoming of their elasticity by the adhesive forces. Quarternary Nitrogen and phosphorous heads make a dipole and provide binding forces for cellular coagulation.

### Example 13: In Vitro Biocompatibility of BioGel and BioPEG

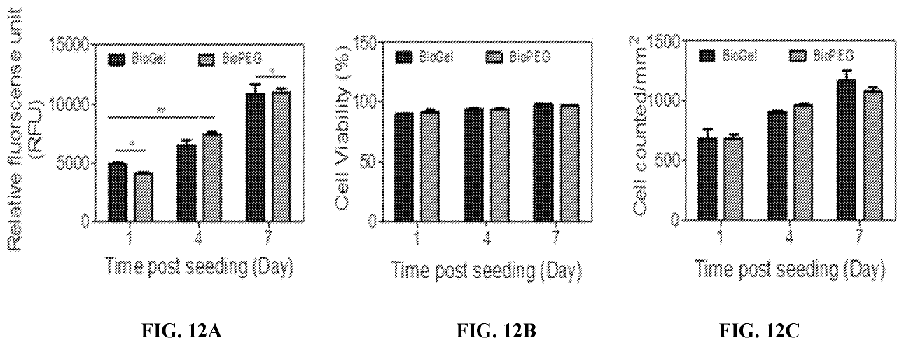

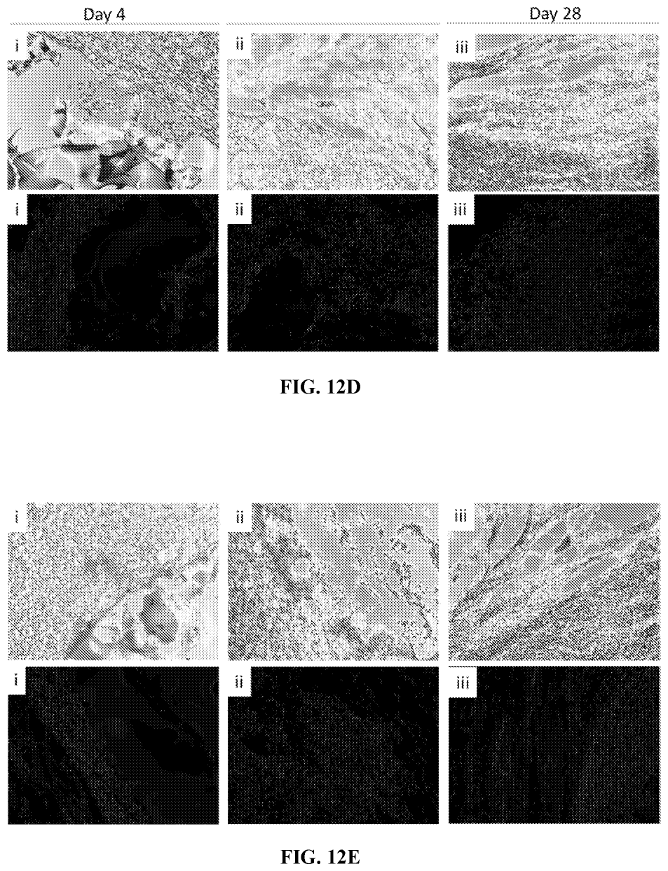

To investigate the biocompatibility of BioGel and BioPEG, 2D cultures in vitro commercial live/dead assay was used. The assay determined the viability of C2C12 cells growing on the BioGel and BioPEG surface over a period of 7 days. Cell attachment and spreading on hydrogels were evaluated through F-actin/DAPI (FIGS. 17A-17D) immunofluorescent staining. The results indicate (FIGS. 12A-12B) that the viability of seeded cells on day 7 was 98.5%±0.5% and 97%±1.0% for BioGel and BioPEG respectively. Cells seeded on the surface of BioGel and BioPEG to exhibit similar viabilities at day one post-seeding. Furthermore, the metabolic activity of the primary cultures quantified by PrestoBlue assay was shown (FIG. 12C) to increase significantly throughout the duration of the culture from 4910.800±180.1301 RFU to 10847.710±797.1749 RFU for BioGel and 4170.601±90.259 RFU to 10954.800±299.443 RFU for BioPEG. These results collectively demonstrate the potential of the bio-adhesive to serve as a biocompatible sealant material that promotes cell adhesion, growth, and proliferation.

### Example 14: In Vivo Biocompatibility and Degradation of BioGel and BioPEG

The in vivo degradation of BioGel and BioPEG synthesized was evaluated after subcutaneous implantation into rats (FIGS. 12E and 12F). Samples were explanted on days 4, 14, and 28 to study the compatibility and degradation. In vivo degradation and morphological changes were characterized by Hematoxylin and eosin (H&E) staining, the staining indicates the presence of the hydrogel until day 4 [FIGS. 12D(i) and 12E(i)] and the tissue architecture revealed that there is no significant host inflammatory response implying that the bio adhesive does not cause any adverse inflammatory responses in both BioGel and BioPEG. Controlling the degradation rate of the sealant is critical to ensure that the sealant material does not completely degrade before tissue healing. Fluorescent immune-histological staining for macrophages (CD68) was used to characterize the local immune response. CD68+ macrophage invasion at the interface between the adhesive and the subcutaneous tissue was observed at day 4 but not at days 28. This observation suggested that the adhesives are efficiently degraded in vivo, through enzymatic hydrolysis of the hydrogel matrix.

### Example 15: Ex Vivo and In Vivo Sealant Performance of BioGel and BioPEG

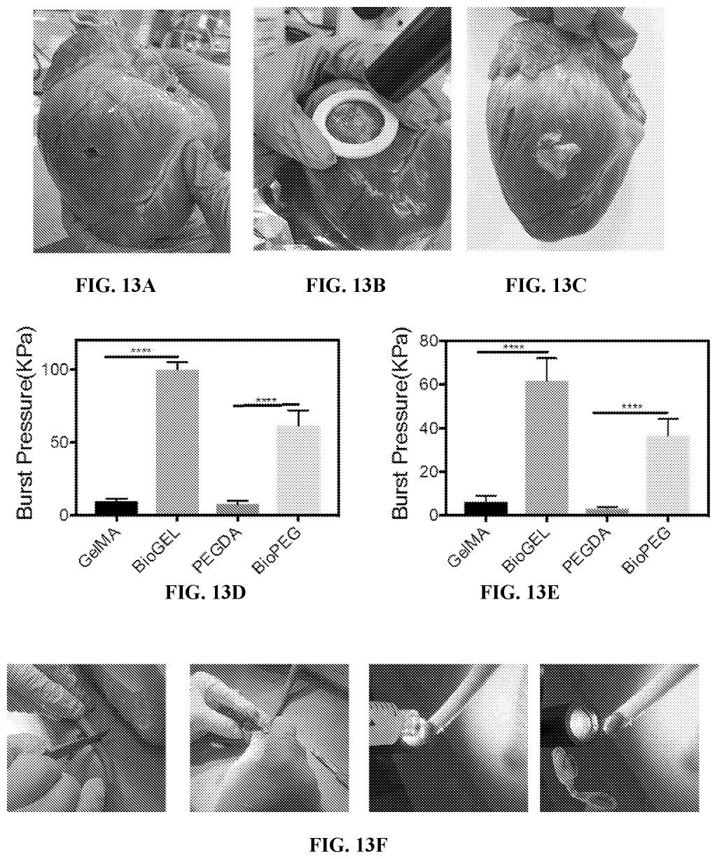

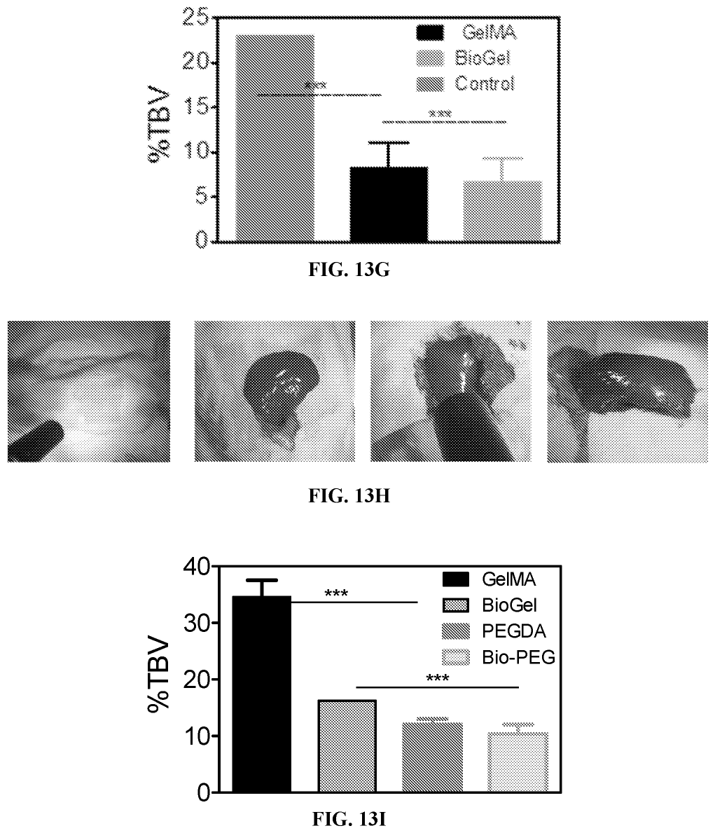

To simulate the ex-vivo properties of the adhesive, burst pressure measurement was carried out on the explanted porcine heart and lung. Specimen chamber was pumped with PBS under a constant flow rate of 2 mL/min, while the pressure was recorded with a pressure gauge. Myocardium specimen and lung before and after burst are shown (FIGS. 13A-13E). The pressure difference between the control and BioGel were tested, 25% (w/v) GelMA exhibited a pressure value of 9.33±1.2 KPa which increased 10 folds with the increasing concentration of BIL to 100±2.89 KPa for BioGel, similarly the difference between 25 (w/v) % PEGDA and BioPEG was 7.33±1.45 KPa and 61.66±6.009 KPa respectively, substantiating the previous values. These results show that BioGel and BioPEG sealant has a significantly greater sealing ability than the other clinically available sealant materials and sutures.

Functional evaluation of BioGel and BioPEG was conducted in a rat model of tail cut and liver laceration (FIGS. 13F-13I). The tail-cut model was performed in 25% GelMA and BioGel, the mean loss of TBV was 0.965%±0.478% and 0.1125%±0.11% respectively which compared to the bleeding controls previously reported as mean loss of TBV 15.4% effectively holds the bleeding. Most of the bleeding occurred in the first 2 minutes but continued steadily until the 8-minute point in both. Whereas in BioGel after the 8^{th} min the bleeding stopped completely. Tail-cut model simulated class I hemorrhagic shock, by developing this set of animal models, a reproducible and quantifiable methods for studying hemostatic properties of the adhesives in vivo is provided. Further to estimate the ability of the adhesive to stop bleeding, the liver laceration model in 25% GelMA and BioGel were performed, the mean loss of TBV was 34.5%±2.9% and 16.2%±4.6% respectively which compared to the bleeding controls previously reported as mean loss of TBV 19.8% effectively holds the bleeding. Most of the bleeding occurred in the first 2 minutes but continued steadily until the 6-minute point in both. But, in BioGel after the 7^{th} min the bleeding stopped completely. Similarly, the mean loss of TBV was 12.06%±0.98% and 9.282%±1.522% for PEGDA and BioPEG respectively indicating that with the conjugation of the BIL the bleeding can be retracted.

The tail-cut is a reproducible model for Class I Hemorrhage in rats. Class I Hemorrhage is defined as blood loss <15% of total blood volume. Both GelMA and BioGel had average percent blood losses of 8.13% and 6.74%, respectively. Blood loss was noticeably slowed after application and polymerization of both compounds. The decrease in rate of blood loss is reinforced by the substantial decrease in percent total blood volume lost between the experimental animals and the controls (15.4% and 22.9%). In both experimental groups, bleeding eventually ceased. In the control animals, oozing continued until euthanasia. The liver laceration model was designed to reproduce Class II Hemorrhage in rats. Class II Hemorrhage is defined as blood loss between 15-30% of total blood volume. The external control (data from Morgan et al., 2015, JAMA Surg. 150(4):316-324) noted a percent total blood volume loss of 19.8%. The wedge resection performed in that study limited the raw surface of liver resected for bleeding. By making only one incision to remove a piece of the liver edge, the surface area exposed for bleeding was minimized. In order to challenge the hemostatic properties of the experimental compounds, a more extensive liver wedge resection was performed. A 1.5 cm by 3 cm piece of the liver was excised, creating a right angle to maximize the surface area of bleeding liver tissue. This more extensive resection lead to a percent total blood loss of 48.72% in the control rat, which would qualify as Class IV Hemorrhage (percent total blood loss >40%). Class IV Hemorrhage leads to profound hemodynamic instability in humans, and almost always requires operative intervention for bleeding cessation. All four compounds in our experimental model successfully stopped bleeding from liver wedge resection. Percent total blood volume lost with GelMA and BioGel was 34.5% and 16.2%, respectively. Percent total blood volume lost with PEGDA and BioPEG was 10.57% and 10.38%, respectively. The ability of these compounds to ensure hemostasis in a Class IV Hemorrhage wound solidifies their reliability as hemostatic agents.

### Example 16

One major component of cell membranes are phospholipids comprising of phosphatidyl choline hydrophilic headgroup, while the hydrophobic tail is comprised of long chains from fatty acids. The hydrophilic heads and hydrophobic heads bunch together form a part of the bilayer cellular membrane. 1,2-dipalmitoyl-glycero-3-phosphatidyl choline (DPPC) has a typical phospholipid structure and is a component of cell membrane. The phosphatidyl choline groups is known to stabilize the cellular membrane bilayer owing to steric effects of this group, in addition to its net charge neutrality, which prevents it from binding to immunological protein oligomers.

The mechanism of adhesion with the BIL modified GelMA and PEGDA polymers can be visualized as per modeled by Yu et. al. (Yu, et al., 2012, Nature Materials 11(5): 468; “Yu, et al.”) via the formation of the strong, electrostatically bound, quadruple as depicted in the FIG. 1 in Yu, et. al. The clubbing together of the hydrophilic heads and bunching together, separately, of the hydrophobic tails causes the lipid membrane bilayer to form hydrophobic interaction zones at the extracellular surface and in the interior of the cell, the hydrophobic interaction zone being in the middle. At the extracellular surface, the mechanism of adhesion is thought to comprise of the interaction between the hydrophobic phosphatidyl choline heads and the choline pendant groups as well as the unreacted carboxyl pendants of the BioGEL structure. This is illustrated in FIG. 1 in Yu, et al., where the two electrostatic ally bound couples are: (1) the negatively charged phosphatidyl moiety from the phospholipid with the positively charged cholinium ion from BioGEL pendant and (2) the cholinium head of the phospatidyl choline from the cellular bilayer's hydrophilic portion and carboxyl anion pendants from the BioGEL polymer. These two pairs form a tightly bound quadruple leading the very high tissue adhesion propensity as seen in the results. While the PEGDA structure does not proffer carboxyl groups to form the quadruple demonstrated in FIG. 1 in Yu, et al., the strong electrostatic interactions between the cholinium of the BIL functionalized PEGDA polymer and phosphatidyl heads of the cellular lipid bilayer, nonetheless, help to maintain high levels of adhesion.

The mechanistic details illustrated have been spectroscopically studied using a Cholinium phosphate-Phosphatidyl choline model by Yu, et al. The presence of the methyl groups on the cholinium ion head are expected to hinder electrostatic interactions. The mechanistic proof of the phosphatidyl choline and cholinium groups have been studied by TOF mass spectrometry using a model of prop-2-ynyle choline phosphate (p-CP) and 1,2-dipalmitoyl-glycero-3-phosphocholine (DPPC), wherein the association product peak between DPPC and cholinium phosphate has been identified at m/z 955.56 amu (M+H). It may bring to argument the possibility of cholinium phosphate homodimers, which were not seen to form in the study—only the heterodimers with DPPC were observed, underscoring the mechanism. Again, no heterodimers were seen by this group in prop-2-ynyle phosphatidyl choline and DPPC models, further underscoring the formation of dimers directing specifically, cellular bilayer—choline phosphate association. Macromolecules result in cellular surface aggregation by proffering adsorptive bridging conformation. Additionally, the membrane glycocalyx is thought to exclude them from the intercellular space causing an osmotic gradient mediated aggregation of cell. The suo motu action of a macromolecule in cellular aggregation is further enhanced in this case with the BIL functionalization mediated adhesive action. As the BIL loading increases, the adhesive interaction becomes stronger. This has been seen in choline phosphate density on carrier polymers with cellular aggregation by Yu, et al. leading to cellular aggregation and close proximity juxtaposition of membranes. These tight junction formation is also expected to foster rapid tissue repair. Thus, a methodical platform to imbibe into a biopolymer, the mechanistic ability to attach to the phospholipid cellular bilayer, an intrinsic requisite for a tissue adhesive has been demonstrated. The illustrated general mechanism encompasses the interactions on the outer surface of the membrane bilayer. Thus, a general bio polymer modification platform for making polymeric tissue adhesives via the incorporation of the BIL functionality in a macromolecule has been presented. This platform induces a strong electrostatic interaction due to the cholinium moieties with the hydrophilic cellular bilayer heads.

In certain embodiments, the development of such a general platform, for converting suitable polymer backbones with the appropriate usage of BIL, allows for the rapid and vast development of biocompatible adhesives, tunable to the property requirement of a given tissue. The adhesives discussed herein with the two polymer families as examples illustrate their hemostatic ability, in vivo compatibility, the ability to prevent microbial infection, allowing for a wide vista of applicability opening up possibilities for enhancing the robustness of surgical and in-field application

The disclosures of each and every patent, patent application, and publication cited herein are hereby incorporated herein by reference in their entirety. While this invention has been disclosed with reference to specific embodiments, it is apparent that other embodiments and variations of this invention can be devised by others skilled in the art without departing from the true spirit and scope of the invention. The appended claims are intended to be construed to include all such embodiments and equivalent variations.

1. A method of treating a wound in a subject in need thereof, the method comprising:
(a) contacting the wound with a composition comprising:
a polymer selected from the group consisting of gelatin methacrylate (GelMa) and poly(ethylene glycol) diacrylate (PEGDA);
choline acrylate; and
at least one photoinitiator; and
(b) exposing the composition to at least one wavelength of light capable of activating the at least one photoinitiator, thereby polymerizing the composition.

2. The method of claim 1, wherein the composition comprises about 1:4 to about 4:1 choline acrylate to polymer.

3. (canceled)

4. The method of claim 1, wherein at least one photoinitiator is selected from the group consisting of eosin Y, 2-hydroxy-2-methylpropiophenone, 2-methyl-4′-(methylthio)-2-morpholinopropiophenone, lithium phenyl-2,4,6-trimethylbenzoylphosphinate (LAP), and 2-hydroxy-4′-(2-hydroxyethoxy)-2-methylpropiophenone (Irgacure).

5. The method of claim 1, wherein the composition further comprises at least one additional compound selected from the group consisting of triethanolamine (TEOA) and N-vinylcaprolactam (VC).

6. (canceled)

7. The method of claim 1, wherein the composition comprises about 10% to about 20% (w/v) choline acrylate.

8. The method of claim 1, wherein the composition comprises about 10% to about 30% (w/v) polymer.

9. The method of claim 4, wherein the composition comprises at least one of:
about 0.1 mM eosin Y;
0.5% (w/v) LAP;
about 1.5% (w/v) TEOA; or
about 1% (w/v) VC.

10. (canceled)

11. (canceled)

12. (canceled)

13. The method of claim 1, wherein the polymerized composition forms an air tight seal or a hemostatic seal on the wound.

14. (canceled)

15. (canceled)

16. The method of claim 13, wherein the polymerized composition forms a seal having a burst pressure of at least 5 kPa.

17. (canceled)

18. (canceled)

19. The method of claim 13, wherein the polymerized composition forms an antimicrobial seal on the wound.

20. The method of claim 19, wherein the polymerized composition forms an antibacterial seal on the wound.

21. The method of claim 13, wherein the polymerized composition forms a seal capable of inhibiting the growth, proliferation and/or survival of at least one bacterial strain selected from the group consisting of Aggregatibacter actinomycetemcomitans, Porphyromonas gingivalis, Prevotella intermedia, Bacteroides forsythus, Campylobacter rectus, Eubacterium nodatum, Peptostreptococcus micros, Staphylococcus intermedius, Pseudomonas aeruginosa, Acinetobacter baumannii and Treponema sp.

22. The method of claim 13, wherein the polymerized composition retains a seal on the wound for at least 28 days.

23. The method of claim 1, which increases the rate of clotting in the wound.

24. The method of claim 1, wherein the wound is in an organ selected from the group consisting of a liver, a lung, a heart, a stomach, an intestine, a pancreas, a kidney, a bladder, an artery, a vein, a skin, a brain, and a joint tissue.

25. The method of claim 1, wherein the composition is contacted with the wound during a surgical procedure.

26. The method of claim 1, wherein the composition is contacted with the wound via injection.

27. The method of claim 1, which further comprises suturing the wound.

28. The method of claim 1, which does not further comprise suturing the wound.

29. The method of claim 1, wherein the subject is a mammal.

30. (canceled)
